(19) United States
(12) Patent Application Publication
(10) Pub. No.: US 2023/0121903 A1
(43) Pub. Date: Apr. 20, 2023

(54) DEVICE AND ANALYSIS METHOD FOR APPRECIATING AND IDENTIFYING SMELLS

(71) Applicant: GEORGE MASON UNIVERSITY, Fairfax, VA (US)

(72) Inventors: Qiliang LI, Fairfax, VA (US); Zhenyi Ye, Centreville, VA (US)

(21) Appl. No.: 17/967,109

(22) Filed: Oct. 17, 2022

Related U.S. Application Data
(60) Provisional application No. 63/256,734, filed on Oct. 18, 2021.

(51) Int. Cl. G06N 3/08; G01N 15/06; G01N 1/24; G01N 1/22

(52) U.S. Cl. CPC G06N 3/08; G01N 15/0656; G01N 1/24; G01N 1/2273; G01N 2015/0046

(57) ABSTRACT

According to an embodiment, it is a system, comprising, a specialized device comprising, a flow sub-system configured for sampling a gas sample, a gas chamber having a gas sensor array comprising a configurable sensor interface, wherein the specialized device is operable to collect an aroma signal from the gas sample, a microcontroller comprising a processor and a memory operable to digitalize the aroma signal to obtain aroma data, store and transfer an aroma data, perform an aroma analysis on the aroma data, and provide a feedback to a user, wherein the system is an aroma evaluation system operable to detect a target aroma in real-time, and wherein the system is operable to interface to at least one of a cloud platform and a smartphone.

### CROSS-REFERENCE TO RELATED APPLICATIONS

This application claims the benefit under 35 U.S.C § 119 of U.S. Provisional Application No. 63/256,734 filed on Oct. 18, 2021, which is hereby incorporated by reference in its entirety.

### FIELD OF THE INVENTION

The present disclosure relates generally to a system for appreciating and identifying smells. More particularly, the present disclosure relates to a system and method for aroma analysis along with providing user feedback. The invention is applicable to various fields including consumer markets for evaluating commodities.

### BACKGROUND

“Metal Oxide (MOX) gas sensors, in particular Metal Oxide Semiconductor (MOS) gas sensors may be applied to various industrial fields and may comprise low cost sensors with respect to devices based on different sensing technologies. MOX gas sensors may exhibit one or more advantages, e.g., a power reduction and low noise with respect to other types of gas sensors. However, they may also exhibit the disadvantage of limited selectivity” [US Publication Number 20200088705A1].

“Existing technologies for detecting VOCs, such as electronic noses, have limitations; for example: sensitivity to oxygen containing compounds, ammonia, carbon-dioxide, high operating temperatures, sensitive to humidity, bulky, complex setup, requirement of controlled environment, limited shelf life, lack of reusability due to permanent denaturing of certain elements upon single exposure to certain VOCs, complex circuitry, and baseline drift, among others. Such technologies can have high operational expenses, maintenance costs, and training costs, as well as complex operational procedures making them unsuitable for large scale deployment. Accordingly, a system and method for detecting VOCs is desired that alleviates limitations of current methods and systems, such as through the application of machine learning techniques to image data.” [US Publication Number 20190317079A1].

“Volatility is the tendency of a substance to vaporize or sublimate. Most compounds are not volatile enough at ambient conditions to be detected by electronic nose sensors. Consequently, there is a need for devices and methods that enhance the volatiles in a sample to improve detection by electronic nose sensors. Moreover, there is a need to fractionate the volatile compounds for detection, analysis, and delivery.” [US Publication Number 20160161459A1].

“More recently, electronic nose (e-nose) systems, have been used as an alternative method of gas detection. E-nose systems are based on sensor arrays coupled with pattern recognition systems. In an e-nose system, the gas sensor array provides a fingerprint response to a given odor; then, a pattern recognition software tool, is used to perform odor identification and discrimination [12-13]. Despite the general success of electronic noses, there are practical challenges in adaptation of this technology: in essence, the inevitable multidimensional drifts of the components of the sensor array result in frequent replacement of the expensive parts and cumbersome recalibrations [14]. Moreover, since general-purpose gas sensors are not selective against different gases, the sensor array used in e-noses is required to have a specific sensor for detecting each target gas. This makes the drift compensation and sensor recalibration even more complicated [15-16].” [US Publication Number 20180120278A1].

Considering the knowledge of a person skilled in the art, there is a need for a system and a device which is easy to handle, can interface with various sensors, can provide results in real-time and offers feedback on user interfaces and alleviates limitations of sensitivity to provide more accurate results.

### SUMMARY

According to an embodiment, it is a system, comprising: a specialized device comprising: a flow sub-system configured for sampling a gas sample; a gas chamber having a gas sensor array comprising a configurable sensor interface; wherein the specialized device is operable to collect an aroma signal from the gas sample; a microcontroller comprising a processor and a memory, operable to: digitalize the aroma signal to obtain aroma data; store and transfer an aroma data; perform an aroma analysis on the aroma data; and provide a feedback to a user; wherein the system is an aroma evaluation system operable to detect a target aroma in real-time; and wherein the system is operable to interface to at least one of a cloud platform and a smartphone.

According to an embodiment of the system, the specialized device is capable of sniffing aroma through at least a first inlet and a second inlet wherein the first inlet is for intake of the target aroma and the second inlet is for intake of air to exhaust the target aroma in the specialized device.

According to an embodiment of the system, the first inlet and the second inlet are controlled by a solenoid valve wherein the solenoid valve is a 3-way solenoid valve.

According to an embodiment of the system, the flow sub-system comprises a flow controller, a pump, and a gas flow sensor.

According to an embodiment of the system, the flow sub-system comprises a configurable air flow scheme through a flow controller for the sampling of the gas sample.

According to an embodiment of the system, the configurable air flow scheme comprises a micro air flow sensor operable to monitor a flow rate of the gas sample through the system.

According to an embodiment of the system, the configurable air flow scheme is operable to multiple patterns of sampling air which creates distinct features for the target aroma.

According to an embodiment of the system, the flow controller is a software reading feedback from the gas flow sensor and calculating a corresponding control signal for the pump.

According to an embodiment of the system, the flow rate is controlled to generate multiple patterns including sinusoid, pulses, and even simulated human breath.

According to an embodiment of the system, the specialized device comprises a heat modulated sampling.

According to an embodiment of the system, the gas sensor array is further integrated with heaters wherein heaters are turned on for a certain amount of time to enhance response.

According to an embodiment of the system, the aroma signal is operable to be analyzed using pattern recognition and artificial intelligence algorithms.

According to an embodiment of the system, the gas sensor array is operable to sense a signal generated by the target aroma.

According to an embodiment of the system, each sensor in the gas sensor array is configured to generate a time series signal with a period of the gas sensor responding and recovery.

According to an embodiment of the system, the aroma signal is sampled and captured by the microcontroller on the specialized device wherein the aroma signal is further conditioned, wherein signal conditioning comprises denoising with discrete wavelet transform, normalizing based on a sensor baseline, extracting features based on signal's magnitude, gradient during sensor response and recovery, and curve fitting parameters.

According to an embodiment of the system, the microcontroller is operable to store the aroma data locally.

According to an embodiment of the system, the microcontroller is operable to store the aroma data locally onto a non-volatile memory card.

According to an embodiment of the system, the microcontroller is further operable to transfer the aroma data to at least one of the cloud platform and a smartphone device.

According to an embodiment of the system, the microcontroller is further operable to transfer the aroma data through at least one of a Wi-Fi and a Bluetooth.

According to an embodiment of the system, the microcontroller is operable to perform the aroma analysis, and a result of identification of the aroma and to return the result to the user in real-time.

According to an embodiment of the system, the aroma analysis comprises detecting information of a breed and the target aroma, wherein the breed comprises one of a different types of target aroma and concentration of certain chemicals in the target aroma.

According to an embodiment of the system, the microcontroller is operable to perform the aroma analysis comprising a pattern recognition algorithm for a target discrimination which is deployed on a standalone device.

According to an embodiment of the system, the microcontroller is operable to perform the aroma analysis comprising a machine learning model aided by an artificial intelligence algorithm powered by a neural network running on the cloud platform, wherein a new test case is operable to be compared to samples in a database upon request of the user.

According to an embodiment of the system, the machine learning model is configured to learn using labeled data using a supervised learning, wherein the supervised learning comprises a logic using at least one of a decision tree, a logistic regression, a k-nearest neighbors, a Naïve Bayes, a random forest, a linear regression, a polynomial regression, a gradient boosting algorithm, and a support vector machine for regression.

According to an embodiment of the system, the machine learning model is configured to learn from real-time data using an unsupervised learning, wherein the unsupervised learning comprises a logic using at least one of a k-means clustering, a hierarchical clustering, a hidden Markov model, and an apriori algorithm.

According to an embodiment of the system, the machine learning model has a feedback loop, wherein an output from a previous step is fed back to the machine learning model in real-time to improve performance and accuracy of an output of a next step.

According to an embodiment of the system, the machine learning model comprises a recurrent neural network model.

According to an embodiment of the system, the machine learning model has a feedback loop, wherein the learning is further reinforced with a reward for each true positive of an output of the system.

According to an embodiment of the system, the microcontroller is operable to perform the aroma analysis wherein pattern recognition results generated locally can form an ensemble with the result generated on the cloud to increase an accuracy of prediction.

According to an embodiment of the system, the configurable sensor interface is designed to accommodate different interfaces, mechanisms, and sizes of the gas sensor array.

According to an embodiment of the system, the configurable sensor interface is a three-level interface comprising a control board, a sensor board, and an interface board.

According to an embodiment of the system, the interface board comprises configurable blocks to create a customizable layout to accommodate various sensor combinations and placement schemes.

According to an embodiment of the system, the configurable sensor interface comprises a first analog to digital converter for an analog sensor and an analog front-end (AFE) and a second analog to digital converter for an electrochemical (EC) sensor.

According to an embodiment of the system, the configurable sensor interface is operable to convert an analog sensor signal to a digital signal.

According to an embodiment of the system, the configurable sensor interface further comprises a humidity sensor and a temperature sensor.

According to an embodiment of the system, the configurable sensor interface further comprises an onboard power light-emitting diode (LED) to indicate working status.

According to an embodiment of the system, the aroma evaluation system is operable to provide a high-level information of the target aroma comprising breed and origin in a case of coffee.

According to an embodiment of the system, the feedback to the user is through an interactive display.

According to an embodiment of the system, the feedback to the user comprises information of a breed and the target aroma, wherein the breed comprises one of a type of target aroma and concentration of certain chemicals in the target aroma.

According to an embodiment, it is a method, comprising: obtaining an aroma signal from a gas sensor, comprising: starting a pump of a flow sub-system to let air flow into a gas chamber of a specialized device comprising a gas sensor array until reaching a steady state; switching a solenoid valve to intake a target aroma which is a gas sample for a certain amount of time; stopping the pump; shutting down the gas chamber, such that molecules in the target aroma fully interact with the gas sensor array; switching the solenoid valve to bring in air by the pump to exhaust the target aroma remaining; reading the aroma signal from the gas sensor; digitalizing the aroma signal to an aroma data; storing and transferring the aroma data; performing an aroma analysis on the aroma data; and providing feedback to a user; wherein the method is an aroma evaluation method operable to detect the target aroma in real-time; and wherein the method is executable on a device that is operable to interface to at least one of a cloud platform and a smartphone.

According to an embodiment of the method, the specialized device is capable of sniffing aroma through at least a first inlet and a second inlet wherein the first inlet is for intake of the target aroma and the second inlet is for intake of air to exhaust the target aroma in the specialized device.

According to an embodiment of the method, the first inlet and the second inlet are controlled by the solenoid valve wherein the solenoid valve is a 3-way solenoid valve.

According to an embodiment of the method, the flow sub-system further comprises a flow controller, and a gas flow sensor along with the pump.

According to an embodiment of the method, the flow sub-system comprises a configurable air flow scheme through a flow controller for sampling of the gas sample.

According to an embodiment of the method, the configurable air flow scheme comprises a micro air flow sensor operable to monitor a flow rate of the gas sample.

According to an embodiment of the method, the configurable air flow scheme is operable to multiple patterns which creates distinct features for the target aroma.

According to an embodiment of the method, the flow controller is a software reading feedback from the gas flow sensor and calculating a corresponding control signal for the pump.

According to an embodiment of the method, the flow rate is controlled to generate multiple patterns including sinusoid, pulses, and even simulated human breath.

According to an embodiment of the method, the gas sensor array of the specialized device comprises a heater modulated sampling.

According to an embodiment of the method, the gas sensor array is further integrated with heaters wherein heaters are turned on for the certain amount of time to enhance response.

According to an embodiment of the method, the aroma signal is operable to be analyzed using pattern recognition and artificial intelligence algorithms.

According to an embodiment of the method, the target aroma is operable to interact with the gas sensor array in the gas chamber and corresponding signals are generated for each sensor.

According to an embodiment of the method, each sensor in the gas sensor array will generate a time series signal with a period of the gas sensor responding and recovering.

According to an embodiment of the method, the aroma signal is sampled and captured by a microcontroller on the specialized device wherein the aroma signal is further conditioned, wherein signal conditioning comprises denoising with discrete wavelet transform, normalizing based on a sensor baseline, extracting features based on signal's magnitude, gradient during sensor response and recovery, and curve fitting parameters.

According to an embodiment of the method, the microcontroller is operable to store the aroma data locally.

According to an embodiment of the method, the microcontroller is operable to store the aroma data locally onto a non-volatile memory card.

According to an embodiment of the method, the microcontroller is further operable to transfer the aroma data to at least one of the cloud platform and a smartphone device.

According to an embodiment of the method, the microcontroller is further operable to transfer the aroma data through at least one of a Wi-Fi and a Bluetooth.

According to an embodiment of the method, the microcontroller is operable to perform the aroma analysis, and to return a result to the user in real-time.

According to an embodiment of the method, the microcontroller is operable to perform the aroma analysis comprising a pattern recognition algorithm for a target discrimination which is deployed on a standalone device.

According to an embodiment of the method, the microcontroller is operable to perform the aroma analysis using artificial intelligence algorithms powered by a neutral network running on the cloud platform, wherein a new test case is operable to be compared to samples in a database upon request of the user.

According to an embodiment of the method, the microcontroller is operable to perform the aroma analysis wherein pattern recognition results generated locally form an ensemble with a result generated on the cloud platform to increase an accuracy of prediction.

According to an embodiment of the method, the aroma analysis comprises information of breed and the target aroma, wherein the breed comprises one of a different types of target aroma and concentration of certain chemicals in the target aroma.

According to an embodiment of the method, a configurable sensor interface is designed to accommodate different interfaces, mechanisms, and sizes of the gas sensor array.

According to an embodiment of the method, the configurable sensor interface is a three-level interface comprising a control board, a sensor board, and an interface board.

According to an embodiment of the method, the interface board comprises configurable blocks to create a customizable layout to accommodate various sensor combinations and placement schemes.

According to an embodiment of the method, the configurable sensor interface comprises a first analog to digital converter for an analog sensor and an analog front-end (AFE) and a second analog to digital converter for an electrochemical (EC) sensor.

According to an embodiment of the method, the configurable sensor interface is operable to convert a sensor analog signal to a digital signal.

According to an embodiment of the method, the configurable sensor interface further comprises humidity sensor and temperature sensor.

According to an embodiment of the method, the configurable sensor interface further comprises an onboard power LED to indicate working status.

According to an embodiment of the method, the aroma evaluation method is operable to provide a high-level information of the target aroma comprising breed and origin in a case of coffee.

According to an embodiment of the method, the feedback to the user is through an interactive display.

According to an embodiment of the method, the feedback to the user comprises information of a breed and the target aroma, wherein the breed comprises one of a different types of target aroma and concentration of certain chemicals in the target aroma.

According to another embodiment, it is a device comprising a flow sub-system configured for sampling a gas sample; a gas chamber having a gas sensor array comprising a configurable sensor interface; wherein the device is operable to collect an aroma signal from the gas sample; a microcontroller comprising a processor and a memory, operable to: digitalize the aroma signal to obtain aroma data; store and transfer an aroma data; perform an aroma analysis on the aroma data; and provide a feedback to a user; wherein the device is an aroma evaluation system operable to detect a target aroma in real-time; and wherein the device is operable to interface to at least one of a cloud platform and a smartphone.

According to another embodiment, it is a non-transitory computer-readable medium having stored thereon instructions executable by a computer system to perform operations comprising: obtaining an aroma signal from a gas sensor, comprising: starting a pump of a flow sub-system to let air flow into a gas chamber of a specialized device comprising a gas sensor array until reaching a steady state; switching a solenoid valve to intake a target aroma which is a gas sample for a certain amount of time; stopping the pump; shutting down the gas chamber with at least two solenoid valves around it, such that molecules in the target aroma fully interact with the gas sensor array; switching the solenoid valve to bring in air by the pump to exhaust the target aroma remaining; reading the aroma signal from the gas sensor; digitalizing the aroma signal to aroma data; storing and transferring the aroma data; performing an aroma analysis on the aroma data; and providing feedback to a user; wherein the instructions executable by the computer system are configured for an aroma evaluation operable to detect the target aroma in real-time; and wherein the instructions are executable on a device that is operable to interface to at least one of a cloud platform and a smartphone.

### BRIEF DESCRIPTION OF THE FIGURES

FIG. 1A shows the structure of the smell sensing device according to an embodiment of the disclosure.

FIG. 1B shows a gas flow mechanism in the smell sensing device according to an embodiment of the disclosure.

FIG. 2 shows a flowchart of the aroma discrimination according to an embodiment of the disclosure.

FIG. 3 shows a system interface hierarchy according to an embodiment of the disclosure.

FIG. 4 shows a pin definition according to an embodiment of the disclosure.

FIG. 5 shows an interface board layout where each block can be one of the three types of sensors according to an embodiment of the disclosure.

FIG. 6 shows a sensor board pinout definition for different types of sensors. C, R, and W for electrochemical (EC) sensors represent Count, Reference and Working electrode, according to an embodiment of the disclosure.

FIG. 7 shows a flow control loop according to an embodiment of the disclosure.

FIG. 8 shows a sniffing scheme simulating human breath according to an embodiment of the disclosure.

FIG. 9 shows a heat modulated sampling scheme, showing heater status, and corresponding sensor response during one sampling cycle according to an embodiment of the disclosure.

FIG. 10 shows a sample sensor response for one sample cycle of the device according to an embodiment of the disclosure.

FIG. 11 shows identification of different coffees from their smells according to an embodiment of the disclosure.

FIG. 12 shows the precise detection and identification of a variety of essential oils according to an embodiment of the disclosure.

FIG. 13A shows a structure of the neural network/machine learning model with a feedback loop.

FIG. 13B shows a structure of the neural network/machine learning model with reinforcement learning.

FIG. 14 shows the system for qualitative and quantitative blood glucose prediction investigated and developed by using an E-Nose system to measure the exhaled breath.

FIG. 15 shows the MOX gas sensor array (left); and design diagram of the system used in the experiment according to an embodiment.

FIG. 16 shows the system diagram of the E-Nose device according to an embodiment.

FIG. 17 shows the MOX gas sensors used in the e-nose system according to an embodiment.

FIG. 18 shows the physical information of recruited participants with evenly distributed age and body mass index (BMI) according to an embodiment.

FIG. 19 shows the electrical signals of all 12 sensors that were measured simultaneously in response to a breath sample and recorded by the E-Nose, according to an embodiment.

FIG. 20 shows the median values of normalized maximum sensor responses versus blood glucose ranges for all sensor features according to an embodiment.

FIG. 21 shows the testing set that was selected by randomly choosing 30% of the samples on each day, according to an embodiment.

FIG. 22 listed the detailed prediction results of Gradient Boosting Tree GBT, according to an embodiment.

FIG. 23 shows the regression performance, according to an embodiment.

FIG. 24A shows the glucose level predicted using exhaled breath versus ground truth obtained by blood tests for SVM regressor (SVR).

FIG. 24B shows the glucose level predicted using exhaled breath versus ground truth obtained by blood tests for Random Forest regressor.

FIG. 24C shows the glucose level predicted using exhaled breath versus ground truth obtained by blood tests for gradient boosting regressor (GBR).

FIG. 25A shows a hybrid regressor decision making diagram where GBT classifier was trained to determine the blood glucose range of data samples; both SVR and GBR were trained to provide refined prediction on different ranges according to an embodiment.

FIG. 25B shows prediction of the hybrid regressor versus ground truth according to an embodiment.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first”, “second”, “third”, “fourth”, and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include” and “have”, and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left”, “right”, “front”, “back”, “top”, “bottom”, “over”, “under”, and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has”, “have”, “having”, or the like are intended to be open-ended terms. Further, the phrase “based on” is intended to mean “based, at least in part, on” unless explicitly stated otherwise.

The terms “couple”, “coupled”, “couples”, “coupling”, and the like should be broadly understood and refer to connecting two or more elements mechanically and/or otherwise. Two or more electrical elements may be electrically coupled together, but not mechanically or otherwise coupled together. Coupling may be for any length of time, e.g., permanent, or semi-permanent or only for an instant. “Electrical coupling” and the like should be broadly understood and include electrical coupling of all types. The absence of the word “removably”, “removable”, and the like near the word “coupled”, and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In several embodiments, “real time” can mean real time less a time delay for processing (e.g., determining) and/or transmitting data. The time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

As used herein, the term component is intended to be broadly construed as hardware, firmware, and/or a combination of hardware and software.

Implementations and all the functional operations described in this specification may be realized in digital electronic circuitry, or in computer software, firmware, or hardware, including the structures disclosed in this specification and their structural equivalents, or in combinations of one or more of them. Implementations may be realized as one or more computer program products i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter affecting a machine-readable propagated signal, or a combination of one or more of them. The term “computing system” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to a suitable receiver apparatus.

The actual specialized control hardware or software code used to implement these systems and/or methods is not limiting of the implementations. Thus, the operation and behavior of the systems and/or methods were described herein without reference to specific software code—it being understood that software and hardware can be designed to implement the systems and/or methods based on the description herein.

A computer program (also known as a program, software, software application, script, or code) may be written in any appropriate form of programming language, including compiled or interpreted languages, and it may be deployed in any appropriate form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, for example without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of digital computer. Generally, a processor will receive instructions and data from a read only memory or a random-access memory or both. Elements of a computer can include a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, optical disks, or solid-state disks. However, a computer need not have such devices.

Moreover, a computer may be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto optical disks; and CD ROM disks, DVD-ROM disks and solid-state disks. The processor and the memory may be supplemented by, or incorporated in, special purpose logic circuitry.

To provide for interaction with a user, implementations may be realized on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any appropriate form of sensory feedback, e.g., visual feedback, auditory feedback, or tactile feedback; and input from the user may be received in any appropriate form, including acoustic, speech, or tactile input.

Implementations may be realized in a computing system that includes a back-end component, e.g., as a data server, or that includes a middleware component, e.g., an application server, or that includes a front-end component, e.g., a client computer having a graphical user interface or a Web browser through which a user may interact with an implementation, or any appropriate combination of one or more such back end, middleware, or front-end components. The components of the system may be interconnected by any appropriate form or medium of digital data communication, e.g., a communication network. Examples of communication networks include a Local Area Network (“LAN”) and a Wide Area Network (“WAN”), e.g., the Internet.

The computing system may include clients and servers. A client and server are generally remote from each other and typically interact through a communication network. The relationship of the client and server arises by virtue of computer programs running on the respective computers and having a client-server relationship to each other.

Embodiments of the present invention may comprise or utilize a special purpose or general-purpose computer including computer hardware. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, solid state disks, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry a desired program code means in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

While this specification contains many specifics, these should not be construed as limitations on the scope of the disclosure or of what may be claimed, but rather as descriptions of features specific to implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and the described program components and systems may generally be integrated together in a single software product or packaged into multiple software products.

Even though particular combinations of features are recited in the claims and/or disclosed in the specification, these combinations are not intended to limit the disclosure of possible implementations. Other implementations are within the scope of the following claims. For example, the actions recited in the claims may be performed in a different order and still achieve desirable results. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

The foregoing disclosure provides illustration and description but is not intended to be exhaustive or to limit the implementations to the precise form disclosed. Modifications and variations are possible considering the above disclosure or may be acquired from the practice of the implementations.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc.

The invention may also be practiced in distributed system environments where local and remote computer systems, which are linked (either by hardwired data links, wireless data links, or by a combination of hardwired and wireless data links) through a network, both perform tasks. In a distributed system environment, program modules may be in both local and remote memory storage devices.

The term “aroma” as used herein refers to one or more volatilized chemical compounds that are generally found in low concentrations and are detected by a sensor. It is also used interchangeably with other terms such as odor, smell, gas, fragrance, volatile organic compound (VOC).

The term “odor sample or smell sample” as used herein refers to any molecule, compound, or substance that can be volatilized for which a smell detection is sought. It is also called a gas sample.

The term “analyte” as used herein is any molecule that can be volatilized and detected by a sensor array.

The term “a gas sensor array” as used herein means a sensor comprising one or more gas sensors.

The term “gas sensor” refers to Metal Oxide Sensors or MOS Gas sensors (also known as gas detectors) are electronic devices that detect and identify different types of gases.

The term “aroma signal” as used herein refers to a “fingerprint” created by a chemical compound either by a single sensor or an array of sensors.

The term “digitize the aroma signal” as used herein means converting the analog signal generated and detected by the sensor array to a digital signal.

The term “aroma data” as used herein means the digital signal

The term “aroma analysis” as used herein refers to any analysis performed on the aroma data.

The term “feedback” as used herein refers to the information or data provided to a user.

Odor, like light, is one of the most important media for human beings to interpret the environment. Aroma analysis is crucial to industries closely related to human health such as food engineering, environment monitoring and medical treatment. Conventional aroma analysis relies heavily on component decomposition of aroma at a molecule level, in which case Solid-Phase Micro Extraction (SPME)-Gas chromatography-mass spectrometry (GC-MS) is used to extract all the chemical components constituting an odor. Lab approach based on GC-MS is accurate and comprehensive, but it splits the process into sampling stage and analysis stage, which is not suitable for real-time odor monitoring. In addition, complicated operation of devices required for lab methods is not convenient for the consumer market who want to evaluate commodities.

The term “specialized device” as used herein refers to a device comprising a configurable sensor interface that is configurable to operate with various types of sensors and is capable of interfacing with a cloud platform and a smartphone.

Therefore, an invention is presented to enable businesses and consumers to know more about products they sell or purchase conveniently and quickly by applying aroma analysis on their own. The invention, in an embodiment, instead of giving a molecule level constitution, provides high level information of target odors, such as breed and origin in the case of coffee.

According to an embodiment, a gas sensor array is used in the invention to sense the signal generated when interacting with target odors. In an embodiment, signals collected by the device are analyzed using pattern recognition and Artificial Intelligence (AI) algorithms to give the final decision.

### The Device

According to an embodiment, a system for aroma evaluation comprises aroma data collection, data storage and transfer, aroma analysis and user feedback. According to an embodiment, it is a specialized device capable of interfacing with cloud platforms and smartphones.

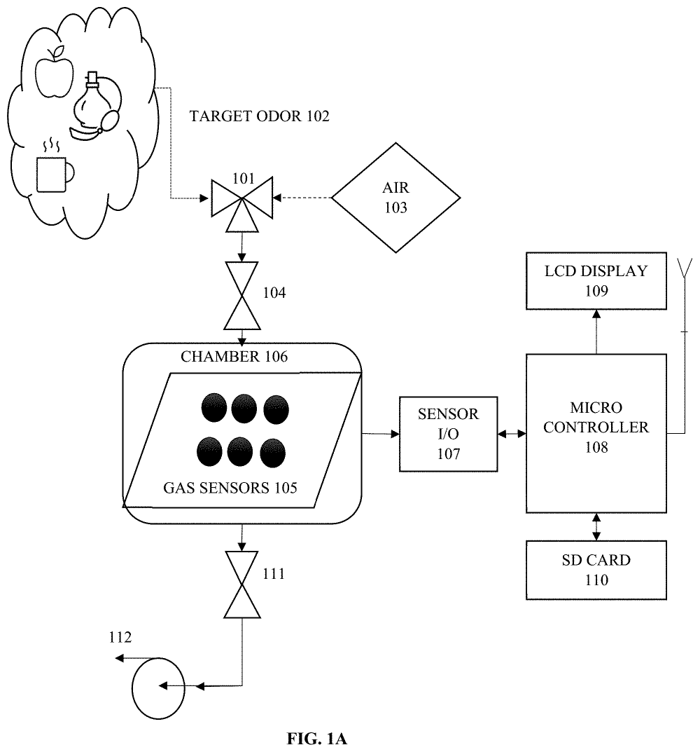

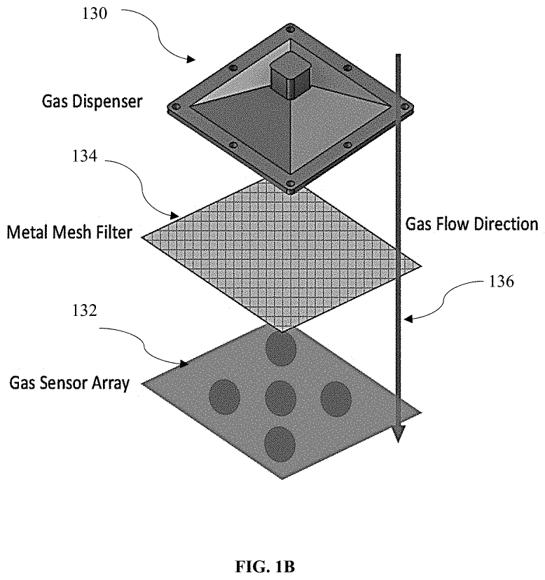

According to an embodiment, the structure of the device is as shown in FIG. 1A. The device comprises two inlets, one is for target aroma as shown at 102 and the other is for air intake as shown at 103 to exhaust remaining target aroma from the device. Both inlets are controlled by a miniature 3-way solenoid valve as shown at 101. The gas chamber valves are controlled by the two solenoid valves 104 and 111 around it, letting the molecules in the target aroma fully interact with the air flow scheme and the gas sensor array. Target aroma will interact with the gas sensor array as shown at 105 in the gas chamber as shown at 106, and corresponding signals are generated on each sensor as shown at sensor input/output (I/O) 107. Signals are sampled and captured by the microcontroller as shown at 108 through the sensor input/output interface on the device in real-time, during which simple signal conditioning will be performed. The microcontroller digitizes the sensor signals. The data collected by the microcontroller can be displayed on a user interface such as an LCD display as shown at 109 and/or can be stored on a memory device such as a Secured Digital (SD) card as shown at 110. The circle as shown at 112 with an arrow is a gas pump driving the gas through the system. The arrows represent the gas flow direction. According to an embodiment, the microcontroller the microcontroller may receive digitized data, wherein digitizing, i.e., Analog to Digital occurs in the configurable sensor interface and is communicated via instrumentation and control system to the microcontroller.

According to an embodiment, during signal conditioning, the collected signals are denoised with discrete wavelet transform; then normalized based on the sensor baseline; after which features are extracted based on signal's magnitude, gradient during sensor response and recovery, and curve fitting parameters. According to an embodiment of the system, the aroma signal is sampled and captured by the microcontroller on the specialized device wherein the aroma signal is further conditioned, wherein signal conditioning comprises denoising with discrete wavelet transform, normalizing based on a sensor baseline, extracting features based on signal's magnitude, gradient during sensor response and recovery, and curve fitting parameters.

According to an embodiment of the method, the aroma signal is sampled and captured by a microcontroller on the specialized device wherein the aroma signal is further conditioned, wherein signal conditioning comprises denoising with discrete wavelet transform, normalizing based on a sensor baseline, extracting features based on signal's magnitude, gradient during sensor response and recovery, and curve fitting parameters. According to an embodiment of the method, the target aroma is operable to interact with the gas sensor array in the gas chamber and corresponding signals are generated for each sensor.

According to an embodiment, working of the device comprises starting the pump to let the air flow into the device, switching on the 3-way solenoid valve to intake target odor for a certain duration or certain amount of time, stopping the pump and shutting the gas chamber by the two solenoid valves around it, letting the molecules in the target odor fully interact with the gas sensor array and switching back the 3-way solenoid valve to let the air in and switching back the pump to exhaust the target odor that is remaining in the chamber.

According to an embodiment, it is a system, comprising: a specialized device comprising: a flow sub-system configured for sampling a gas sample; a gas chamber having a gas sensor array comprising a configurable sensor interface; wherein the specialized device is operable to collect an aroma signal from the gas sample; a microcontroller comprising a processor and a memory, operable to: digitalize the aroma signal to obtain aroma data; store and transfer an aroma data; perform an aroma analysis on the aroma data; and provide a feedback to a user; wherein the system is an aroma evaluation system operable to detect a target aroma in real-time; and wherein the system is operable to interface to at least one of a cloud platform and a smartphone.

According to an embodiment of the system, the first inlet and the second inlet are controlled by a solenoid valve wherein the solenoid valve is a 3-way solenoid valve.

According to an embodiment of the system, the gas sensor array is operable to sense a signal generated by the target aroma.

According to another embodiment, it is a device comprising a flow sub-system configured for sampling a gas sample; a gas chamber having a gas sensor array comprising a configurable sensor interface; wherein the device is operable to collect an aroma signal from the gas sample; a microcontroller comprising a processor and a memory, operable to: digitalize the aroma signal to obtain aroma data; store and transfer an aroma data; perform an aroma analysis on the aroma data; and provide a feedback to a user; wherein the device is an aroma evaluation system operable to detect a target aroma in real-time; and wherein the device is operable to interface to at least one of a cloud platform and a smartphone.

According to another embodiment, it is a non-transitory computer-readable medium having stored thereon instructions executable by a computer system to perform operations comprising: obtaining an aroma signal from a gas sensor, comprising: starting a pump of a flow sub-system to let air flow into a gas chamber of a specialized device comprising a gas sensor array until reaching a steady state; switching a solenoid valve to intake a target aroma which is a gas sample for a certain amount of time; stopping the pump; shutting down the gas chamber with at least two solenoid valves around it, such that molecules in the target aroma fully interact with the gas sensor array; switching the solenoid valve to bring in air by the pump to exhaust the target aroma remaining; reading the aroma signal from the gas sensor; digitalizing the aroma signal to aroma data; storing and transferring the aroma data; performing an aroma analysis on the aroma data; and providing feedback to a user; wherein the instructions executable by the computer system are configured for an aroma evaluation operable to detect the target aroma in real-time; and wherein the instructions are executable on a device that is operable to interface to at least one of a cloud platform and a smartphone.

According to an embodiment, it is a method, comprising: obtaining an aroma signal from a gas sensor, comprising: starting a pump of a flow sub-system to let air flow into a gas chamber of a specialized device comprising a gas sensor array until reaching a steady state; switching a solenoid valve to intake a target aroma which is a gas sample for a certain amount of time; stopping the pump; shutting down the gas chamber, such that molecules in the target aroma fully interact with the gas sensor array; switching the solenoid valve to bring in air by the pump to exhaust the target aroma remaining; reading the aroma signal from the gas sensor; digitalizing the aroma signal to an aroma data; storing and transferring the aroma data; performing an aroma analysis on the aroma data; and providing feedback to a user; wherein the method is an aroma evaluation method operable to detect the target aroma in real-time; and wherein the method is executable on a device that is operable to interface to at least one of a cloud platform and a smartphone. According to an embodiment of the method, the first inlet and the second inlet are controlled by the solenoid valve wherein the solenoid valve is a 3-way solenoid valve.

FIG. 1B shows a gas flow mechanism in the smell sensing device according to an embodiment of the disclosure. As shown in FIG. 1B, the gas will go through a metal mesh filter 134 such that molecules are distributed evenly in the chamber. In each sniffing, the gas is dispensed through the gas dispenser 130, through the mesh filter 134, and onto the gas sensor array 132, in which sensors interact with the incoming gas simultaneously. The gas flow direction is vertical as shown at 136 to the sensor array 132. In an embodiment, the gas pipeline structure as shown in FIG. 1B is hosted inside of the gas chamber (shown as 106 in FIG. TA). The purpose of the structure is to ensure even distribution of gas inside the chamber and have sensors sufficiently interact with the gas.

According to an embodiment of the system, the specialized device is capable of sniffing aroma through at least a first inlet and a second inlet wherein the first inlet is for intake of the target aroma and the second inlet is for intake of air to exhaust the target aroma in the specialized device.

According to an embodiment of the method, the specialized device is capable of sniffing aroma through at least a first inlet and a second inlet wherein the first inlet is for intake of the target aroma and the second inlet is for intake of air to exhaust the target aroma in the specialized device.

The pipeline of gas flow improves the measurement consistency. A gas flow mechanism is designed to serve two purposes: First, it ensures all sensors respond to gas flow change simultaneously, and second, it ensures the gas molecules are evenly distributed in the gas chamber in each measurement.

The flowchart for the smell analysis and detection, according to an embodiment, is shown in FIG. 2. Shown as a test case, the process comprises data collection as shown in column 200-1, data storage as shown in column 200-2 and data analysis as shown in column 200-3.

Data collection as shown at 200-1: Data collection is performed with a device capable of sniffing odor, i.e., capable of receiving smell signals. The process will begin with the start test step as shown at 201 to let the air flow into the device chamber as shown at 202. The air flow is performed for a preset duration and is checked continuously at step 203 to stop the airflow after the preset duration. This airflow will exhaust any gases that were present in the device chamber.

Then, the target odor is let into the smell chamber as shown at 204 for a preset duration and is checked continuously at step 205. Air flow is let into the chamber at 206. The chamber comprises gas sensors where the target odor interacts with the gas sensors producing sensor signals (smell signals).

Data storage and transfer as shown at 202-2: A microcontroller digitizes the sensor signals. When the test data is ready for analysis as shown at 210, the microcontroller will store the data locally onto a Secure Digital (SD) card attached as shown at 208. In addition, data will also be transferred to cloud platforms as shown at 209 through Wi-Fi®, or to a smartphone device as shown at 207 through Bluetooth®.

According to an embodiment of the system, the microcontroller is further operable to transfer the aroma data to at least one of the cloud platform and a smartphone device. According to an embodiment of the system, the microcontroller is further operable to transfer the aroma data through at least one of a Wi-Fi® and a Bluetooth®.

According to an embodiment of the method, the microcontroller is further operable to transfer the aroma data to at least one of the cloud platform and a smartphone device.

According to an embodiment of the method, the microcontroller is further operable to transfer the aroma data through at least one of a Wi-Fi® and a Bluetooth®.

According to an embodiment of the system, the microcontroller is operable to store the aroma data locally. According to an embodiment of the system, the microcontroller is operable to store the aroma data locally onto a non-volatile memory card.

According to an embodiment of the method, the microcontroller is operable to store the aroma data locally. According to an embodiment of the method, the microcontroller is operable to store the aroma data locally onto a non-volatile memory card.

Data analysis as shown at 200-3: The aroma analysis is performed on the microcontroller and the result will be displayed to the user immediately in real-time, including the information such as the breed of the target, through an LCD display. The breeds are different types of target aroma.

For instance, essential oils of different types include orange, mint, cedar, etc., each of these being breeds. “Breeds” could also be the concentration of certain chemicals in the aroma, e.g., the concentration of alcohol, benzene, etc., in a target odor. The corresponding test data is fetched from a cloud platform or from the SD card for further analysis as shown at 211. The analysis is done in a twofold manner. First manner, a pattern recognition algorithm for target discrimination is deployed on the standalone device and is performed by the Micro Controller Unit (MCU) as shown at 212. The second manner, upon user's request as shown at 213, the corresponding test data is fetched from a cloud platform or from the SD card for further analysis as shown at 211 and Artificial Intelligence (AI) algorithms powered by Neutral Network (NN) are run in real-time on the cloud platform as shown at 214, where the new test case can be compared to the samples in the database and the result is displayed as shown at 215 to a user interface such as an LCD. Pattern recognition results generated locally can form an ensemble with the result generated on the cloud to increase real-time NN prediction accuracy.

According to an embodiment of the system, the aroma signal is operable to be analyzed using pattern recognition and artificial intelligence algorithms. According to an embodiment of the system, the microcontroller is operable to perform the aroma analysis comprising a pattern recognition algorithm for a target discrimination which is deployed on a standalone device.

According to an embodiment of the system, the microcontroller is operable to perform the aroma analysis, and a result of identification of the aroma and to return the result to the user in real-time.

According to an embodiment of the method, the aroma signal is operable to be analyzed using pattern recognition and artificial intelligence algorithms. According to an embodiment of the method, the microcontroller is operable to perform the aroma analysis using artificial intelligence algorithms powered by a neutral network running on the cloud platform, wherein a new test case is operable to be compared to samples in a database upon request of the user. According to an embodiment of the method, the microcontroller is operable to perform the aroma analysis comprising a pattern recognition algorithm for a target discrimination which is deployed on a standalone device. According to an embodiment of the method, the microcontroller is operable to perform the aroma analysis, and to return a result to the user in real-time.

Artificial Intelligence for Aroma Analysis: According to an embodiment of the disclosure, the algorithm stage comprises a neural network using one or more trained models and/or a random forest using the one or more trained models.

According to an embodiment, a 1-D convolutional neural network (1D-CNN) is introduced to efficiently process the time-series signals from an electronic nose system, E-Nose. A CNN consists of multiple convolutional layers. Each layer has a set of kernel filters, and the output of the layer is calculated by sliding filters along time direction. According to an embodiment, E-Nose generates unique patterns (breath prints) while measuring the VOCs profile of human breath.

According to an embodiment, a light-weight convolutional neural network model is trained on a computer. The trained model is implemented on the microcontroller. In an embodiment, it is implemented in C++, which can load parameters from computer models and make predictions directly on a microcontroller without the assistance of a network.

The model takes multidimensional gas sensor array time-series data as the input and makes predictions on the property of the gas sample. According to an embodiment, the model is trained in a way that it can perform both qualitative analysis (decide the type of gas sample) and quantitative analysis (decide certain property values of gas source such as concentration, pH values, etc.).

A convolution layer accepts a multichannel one-dimensional signal, convolves it with each of its multichannel kernels, and stacks the results together into a new multichannel signal that it passes on to the next layer. According to an embodiment, a model is tuned to improve performance on the analysis, by data preparation, number of filters and size of kernels.

An artificial neural network is a parameterized statistical model, in which several logistic regressions are combined nonlinearly. Such systems learn to perform tasks by considering examples, generally without being programmed with any task-specific rules. A neural network is based on a collection of connected nodes called artificial neurons. Each connection can transmit a signal from one artificial neuron to another. An artificial neuron that receives a signal can process it and then signal additional artificial neurons connected to it. The structure of the nodes, or the hyper-parameters, of a neural network is predefined by a model, and the parameters of the connections are found by training the neural network. Structure and the corresponding parameters form a trained model for the respective neural network.

A random forest is a learning method for classification, regression and other tasks that operates by constructing a multitude of decision trees at training time and outputting the class that is the mode of the classes (classification) or mean prediction (regression) of the individual trees.

According to embodiments of the disclosure, the one or more trained models comprise one or more trained multiple-output models having a plurality of outputs, wherein for all the gas sensors the output values for the respective gas sensor are created by using one of the trained multiple-output models at the algorithm stage, wherein each of the output values is created at a different output of the plurality of outputs.

According to embodiments of the disclosure, for each of the gas sensors, the one or more trained models comprise one or more trained single-output models having a single output, wherein the output values for the different gas sensors are created by using different single-output models of the trained single-output models at the algorithm stage.

According to embodiments of the disclosure, the preprocessing block comprises a baseline manipulation stage configured for transforming the signal samples of each of the gas sensors into a relative resistance change according to a baseline of the signal samples of the respective gas sensor.

Feature extraction and pattern recognition techniques based on Artificial Neural Networks have been used for classifying the shape of the R−Iₕ curves (Resistance−Current) and potentially they can be used for discovering the co-presence of multiple gases in the environment.

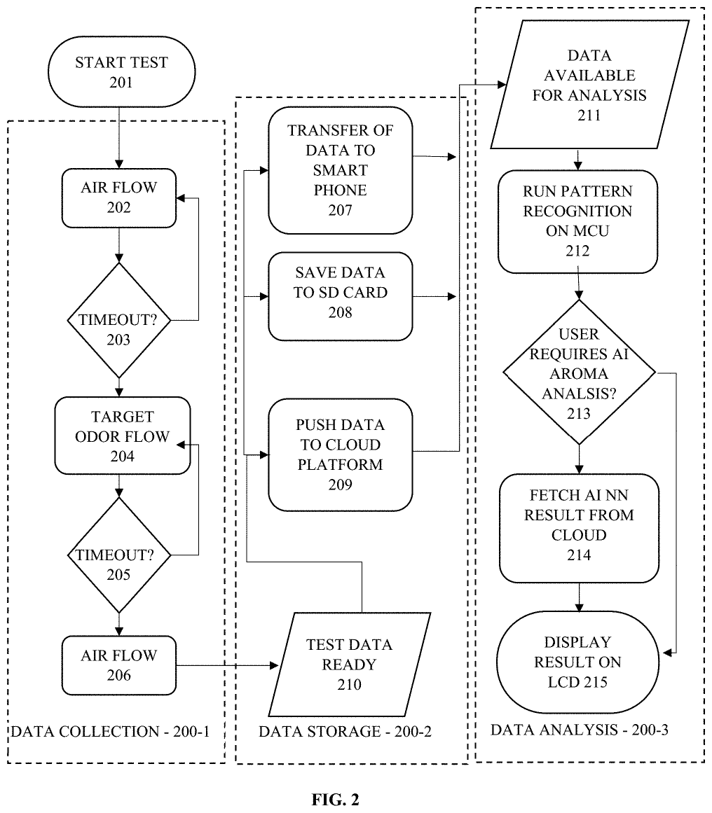

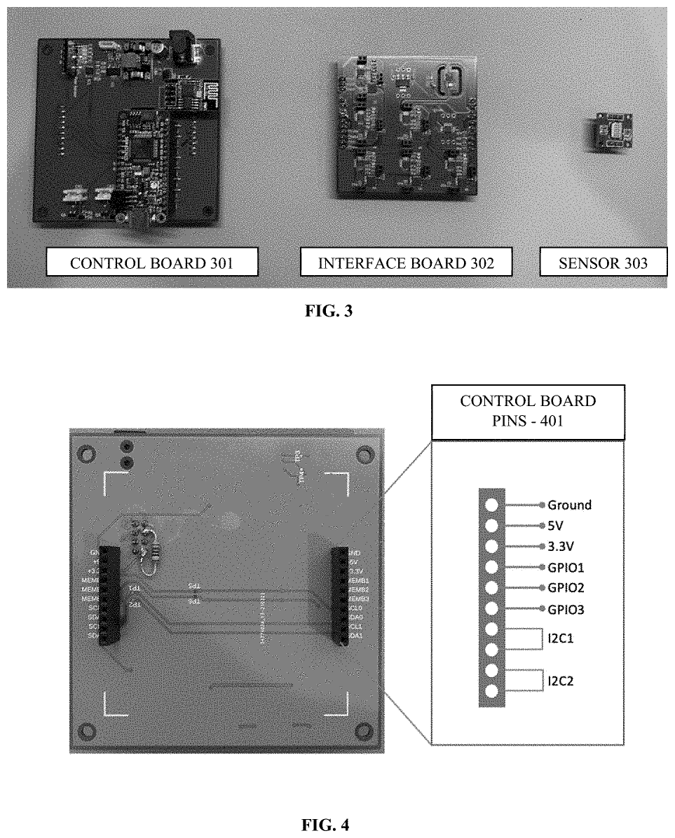

Configurable sensor interface: To accommodate gas sensors of different interfaces, mechanisms and sizes, a three-level gas sensor interface is designed as shown in FIG. 3. The configurable sensor interface can be easily modified for different sensor combinations. The control board as shown at 301 and sensor as shown at 303 are interfaced through a separate interface board as shown at 302, which converts any sensor signals to digital signals. In an embodiment, the sensor 303 is the sensor array comprising plurality of sensors. In an embodiment, the control board as shown at 301 and the interface board 302 may be along with the sensor 303 in the gas chamber. In an embodiment, the control board as shown at 301 and the interface board 302 may be outside the gas chamber.

Control board bus: The control board 301 is shown in FIG. 4. The control board reads digital signals from the interface board through the Inter-Integrated Circuit (I2C) protocol, and it can alternatively read serial signals if sensors have a microcontroller embedded. The control board pins are shown at 401. The control board has an Inter-Integrated Circuit (I2C), which is a bus interface connection protocol incorporated into devices for serial communication. The bus by default has two sets of I2C pins, 6 general purpose inputs/outputs (3 on each side) and power of both 5V and 3.3V as shown at 401. The General-Purpose Input/Output (GPIO)s are used to control sensor specific behaviors (address selections, heater control). GPIO is a standard interface used to connect microcontrollers to other electronic devices. For example, it can be used with sensors, diodes, displays, and System-on-Chip modules.

Interface board: As shown in FIG. 5, an interface board is designed to convert any signals from sensors with different standards to digital signals comprising configurable blocks. In an embodiment, it comprises 8 configurable blocks shown as 501, 502, 503, 504, 505, 506, 507, and 508 that can be moved around during layout to accommodate for different sensor combinations and placements. According to an embodiment, the interface board is of size 6.8×6.8 cm and each block is of size 2×2 cm, and the blocks are arranged into 3×3 grids. In addition to interchangeable sensor blocks, it comprises a humidity sensor and a temperature sensor. In an embodiment, a 2×2 cm area from the interchangeable sensor blocks is left to accommodate the humidity sensor and the temperature sensor. There are different peripherals associated with each block depending on the type of gas sensors. Each analog sensor is associated with an Analog to Digital Converter (ADC); each electrochemical (EC) sensor is associated with an analog frontend (AFE) and an ADC; while a digital sensor does not require Analog to Digital (A-D) conversion and there are no peripheral circuits needed. The same block and its peripherals can be used for sensors of the same type interchangeably, regardless of the sensor's manufacturer, pinouts, and size.

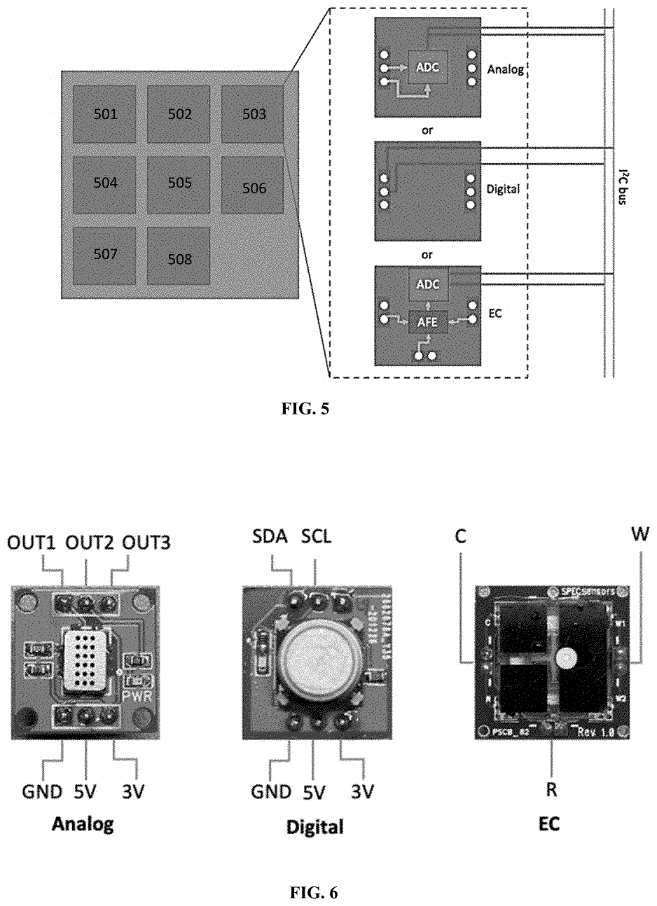

Sensor board: According to an embodiment, all sensor boards are designed to be the same form factor (2×2 cm). Digital sensor and analog sensor boards have the same pin layouts while EC gas sensor is slightly different. The detailed pin definition is shown in FIG. 6. All sensor boards are designed to follow the same pinout standard and both analog and digital sensor boards have onboard power Light Emitting Diode (LED) to indicate its working status. FIG. 6 shows labels, Out1, Out2, Out3 which are sensor signal outputs; GND as electrical ground; 5V, 3V for power supply voltage of 5V and 3V; Serial Data Pin (SDL) for data pin from I²C bus; Serial Clock Pin (SCL) for clock pin from I²C bus; C for counter electrode; W for working electrode and R for reference electrode.

According to an embodiment of the system, the configurable sensor interface is designed to accommodate different interfaces, mechanisms, and sizes of the gas sensor array. According to an embodiment of the system, the configurable sensor interface is a three-level interface comprising a control board, a sensor board, and an interface board.

According to an embodiment of the system, the interface board comprises configurable blocks to create a customizable layout to accommodate various sensor combinations and placement schemes. According to an embodiment of the system, the configurable sensor interface comprises a first analog to digital converter for an analog sensor and an analog front-end (AFE) and a second analog to digital converter for an electrochemical (EC) sensor. According to an embodiment of the system, the configurable sensor interface is operable to convert an analog sensor signal to a digital signal. According to an embodiment of the system, the configurable sensor interface further comprises a humidity sensor and a temperature sensor. According to an embodiment of the system, the configurable sensor interface further comprises an onboard power light-emitting diode (LED) to indicate working status.

According to an embodiment of the method, a configurable sensor interface is designed to accommodate different interfaces, mechanisms, and sizes of the gas sensor array. According to an embodiment of the method, the configurable sensor interface is a three-level interface comprising a control board, a sensor board, and an interface board.

According to an embodiment of the method, the interface board comprises configurable blocks to create a customizable layout to accommodate various sensor combinations and placement schemes. According to an embodiment of the method, the configurable sensor interface comprises a first analog to digital converter for an analog sensor and an analog front-end (AFE) and a second analog to digital converter for an electrochemical (EC) sensor. According to an embodiment of the method, the configurable sensor interface further comprises humidity sensor and temperature sensor. According to an embodiment of the method, the configurable sensor interface further comprises an onboard power LED to indicate working status.

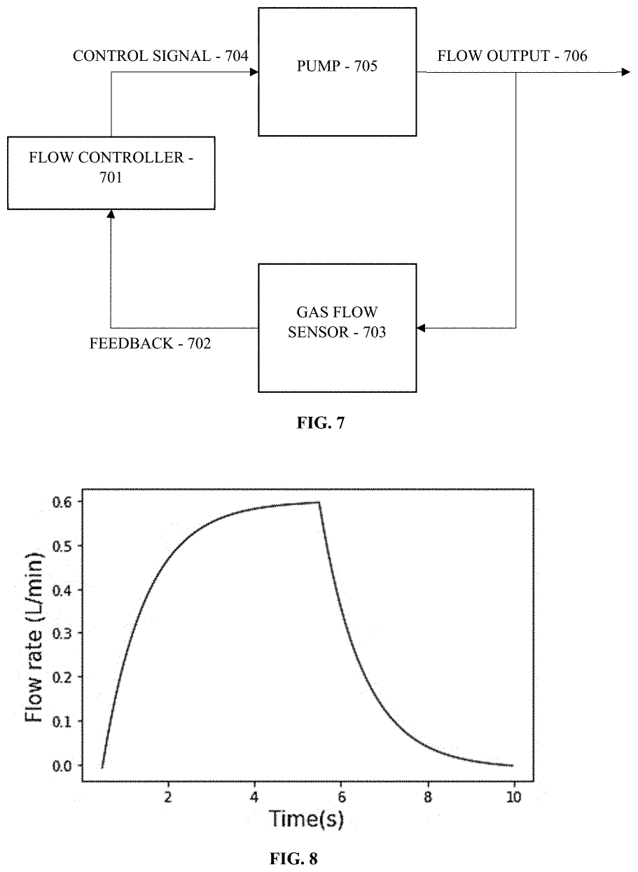

Configurable Sniffing Scheme: During the gas sampling phase as referred to in FIG. 2, a micro air flow sensor will be used for monitoring the flow rate of the gas through the system. Instead of maintaining the consistent flow rate for sampling, the air flow scheme is configured to different patterns, which creates distinct features for odors. According to an embodiment, as shown in FIG. 7, a flow controller as shown at 701 is a software reading feedback as shown at 702 from gas flow sensor as shown at 703 and calculates the corresponding control signal as shown at 704 for the pump as shown at 705 which controls the flow output at 706.

According to an embodiment of the system, the flow sub-system comprises a flow controller, a pump, and a gas flow sensor. According to an embodiment of the system, the configurable air flow scheme comprises a micro air flow sensor operable to monitor a flow rate of the gas sample through the system. According to an embodiment of the system, the flow controller is a software reading feedback from the gas flow sensor and calculating a corresponding control signal for the pump.

According to an embodiment of the method, the flow sub-system further comprises a flow controller, and a gas flow sensor along with the pump. According to an embodiment of the method, the flow sub-system comprises a configurable air flow scheme through a flow controller for sampling of the gas sample. According to an embodiment of the method, the configurable air flow scheme comprises a micro air flow sensor operable to monitor a flow rate of the gas sample.

According to an embodiment of the method, the configurable air flow scheme is operable to multiple patterns which creates distinct features for the target aroma. According to an embodiment of the method, the flow controller is a software reading feedback from the gas flow sensor and calculating a corresponding control signal for the pump.

The flow rate can be controlled to generate multiple patterns including sinusoid, pulses, and even simulated human breath as shown in FIG. 8. Since different sniffing patterns capture distinct features of the same odor, using multiple sniffing schemes during odor identification will introduce extra robustness.

The sniffing pattern is generated and modulated using a feedback loop comprising a Proportional Integral Derivative (PID) controller, pump, and gas flow sensor. The sniffing pattern control features two major advantages compared to simple step sniffing. Firstly, sniffing pattern control can generate richer and more distinct patterns of various gases. Gas sensors, depending on their body porosity and surface-to-body ratio, interact with target gas at a different speed when gas concentration changes. By adjusting the flow rate of gas through the gas chamber, the concentration of gases at specific time points can be modulated, and the signals generated from gas sensors are a combination of sensor responses at different gas concentration levels. Secondly, sniffing pattern control can alleviate sensor saturation. When a single pulse of gas is injected into the gas chamber, gas molecules are fully absorbed into the gas sensor and make it difficult for sensors to recover. The problem is addressed using sniffing pattern control to gradually increase the flow rate and only hold maximum flow rate for a short period of time.

In an embodiment, the sniffing pattern may be a sinusoidal, a human breath pattern, etc. For instance, the flow rate v as a function of time t is v=100|sin (4πt)|. The pattern indicates a sinusoidal sniffing pattern with period is and peak flow rate of 100 sccm, where s stands for seconds and sccm stands for standard cubic centimeter per minute.

According to an embodiment of the system, the flow sub-system comprises a configurable air flow scheme through a flow controller for the sampling of the gas sample. According to an embodiment of the system, the configurable air flow scheme is operable to multiple patterns of sampling air which creates distinct features for the target aroma. According to an embodiment of the system, the flow rate is controlled to generate multiple patterns including sinusoid, pulses, and even simulated human breath.

According to an embodiment of the method, the flow rate is controlled to generate multiple patterns including sinusoid, pulses, and even simulated human breath.

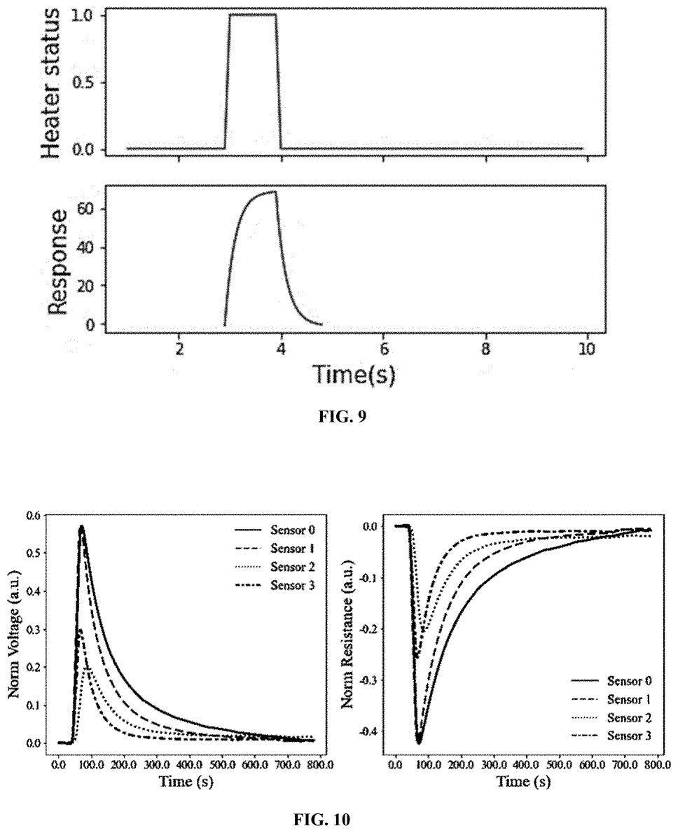

Heater modulated sampling: According to an embodiment, in addition to the changing sniffing scheme, another sampling method is made possible to the sensors with integrated heater modulated sampling. Sensors without heaters will have little response towards target gas unless being heated. At the beginning of sampling, the heaters are turned off and a certain volumes of target gas is pumped into the chamber. Then the chamber valves are shut, and the heaters are turned on for a certain amount of time so that the enhanced responses can be recorded as shown in FIG. 9. According to an embodiment, the duration could be between 1 s to 10 s. By switching on and off the heater, similar responses can be achieved with faster sensor recovery. Curve 901 shows the heater switched on (when heater status is at value 1.0) and switched off (when heater status is at value 0.0). The signal response of a sensor corresponding to a sensor is shown by curve 902 in FIG. 9. According to an embodiment of the system, the specialized device comprises a heat modulated sampling. According to an embodiment of the system, the gas sensor array is further integrated with heaters wherein heaters are turned on for a certain amount of time to enhance response.

According to an embodiment of the method, the gas sensor array of the specialized device comprises a heater modulated sampling. According to an embodiment of the method, the gas sensor array is further integrated with heaters wherein heaters are turned on for the certain amount of time to enhance response.

### EXAMPLES

Case Study 1: Coffee Identification: Coffee aroma varies with respect to different breeds, origin, and roasting approaches. Identifying in real-time, the category and quality of coffee has great demands from coffee planters, coffee shops and consumers to evaluate the commodity.

According to a research study, coffee aroma is itself a complex compound with tens of chemicals falling into categories of aldehydes, pyrazines, ketones, phenols, acids, and heterocyclic N. Coffee varying in origin, breed, and roast methods has distinct distributions of those chemicals, which results in different coffee aroma sensed by a human being.

In a cycle of sampling, each gas sensor in the gas sensor array will generate a time series signal with a period of sensor responding and recovery. Sensor signals start to change when the target aroma is interacting with the sensor array and recovery when air is brought in to exhaust the target aroma. FIG. 10 shows a testing result for a coffee sample with a 4-sensor gas sensor array.

Each of the graphs in FIG. 10 is a response of the sensor, in voltage and resistance, when exposed to a coffee sample. Coffee samples differing in breed, reflect different combinations of patterns on the signals, which can be used to distinguish one category of coffee from another. A breed comprises one of a different types of target aroma and concentration of certain chemicals in the target aroma. According to an embodiment of the system, the aroma evaluation system is operable to provide a high-level information of the target aroma comprising breed and origin in a case of coffee. According to an embodiment of the system, the aroma analysis comprises detecting information of a breed and the target aroma, wherein the breed comprises one of a different types of target aroma and concentration of certain chemicals in the target aroma. According to an embodiment of the system, the feedback to the user is through an interactive display. According to an embodiment of the system, the feedback to the user comprises information of a breed and the target aroma, wherein the breed comprises one of a type of target aroma and concentration of certain chemicals in the target aroma.

According to an embodiment of the method, the aroma evaluation method is operable to provide a high-level information of the target aroma comprising breed and origin in a case of coffee. According to an embodiment of the method, the aroma analysis comprises information of breed and the target aroma, wherein the breed comprises one of a different types of target aroma and concentration of certain chemicals in the target aroma. According to an embodiment of the method, the feedback to the user comprises information of a breed and the target aroma, wherein the breed comprises one of a different types of target aroma and concentration of certain chemicals in the target aroma. According to an embodiment of the method, the configurable sensor interface is operable to convert a sensor analog signal to a digital signal. According to an embodiment of the method, the feedback to the user is through an interactive display.

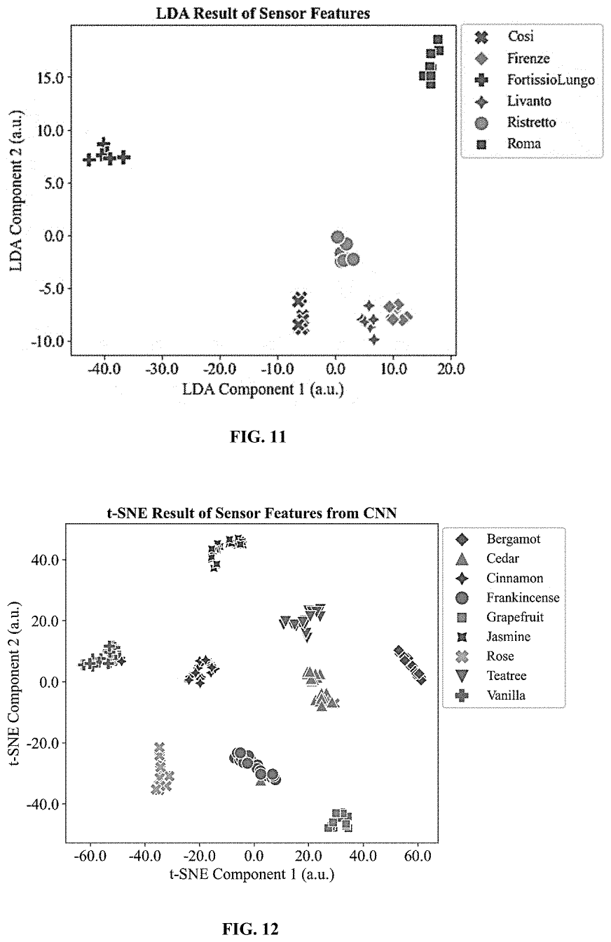

The result of coffee discrimination is given in FIG. 11. Six different coffees can be differentiated from each other by analyzing their pattern extracted from time series signals. In the similar manner, other embodiments include odors that can be differentiated in fruit and fragrance markets. According to another embodiment, various fragrance samples were analyzed by the device and were identified and classified as shown in FIG. 12. FIG. 11 and FIG. 12, the X and Y axes are noted as arbitrary units (a.u.) as there is no unit associated. According to an embodiment of the system, each sensor in the gas sensor array is configured to generate a time series signal with a period of the gas sensor responding and recovery. According to an embodiment of the method, each sensor in the gas sensor array will generate a time series signal with a period of the gas sensor responding and recovering.

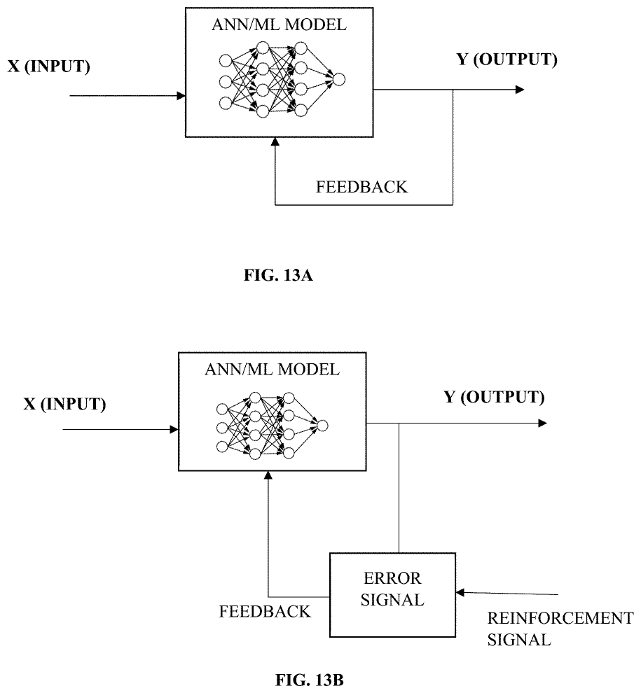

FIG. 13A shows a structure of the neural network or machine learning model with a feedback loop. Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The model may learn from the data. Learning can be supervised learning and/or unsupervised learning and may be based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer may predict or detect a target aroma based on the input data.

In an embodiment, ANN's may be a Deep-Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback. The variations of weights in the hidden layer(s) will be adjusted to fit the expected outputs better while training the model. This will allow the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the Artificial Intelligence/Machine Learning (AI/ML) model is trained well, with large sets of labeled data and concepts, after a while, the models' performance may decline while adding new, unlabeled input due to many reasons which include, but not limited to, concept drift, recall precision degradation due to drifting away from true positives, and data drift over time. A feedback loop to the model keeps the AI results accurate and ensures that the model maintains its performance and improvement, even when new unlabeled data is assimilated. A feedback loop refers to the process by which an AI model's predicted output is reused to train new versions of the model.

Initially, when the AI/ML model is trained, a few labeled samples comprising both positive and negative examples of the concepts (e.g., target aroma) are used that are meant for the model to learn. Afterward, the model is tested using unlabeled data. By using, for example, deep learning and neural networks, the model can then make predictions on whether the desired concept/s (for e.g., target aroma that needs to be detected) are in unlabeled images. Each image is given a probability score where higher scores represent a higher level of confidence in the models' predictions. Where a model gives an image a high probability score, it is auto-labeled with the predicted concept. However, in the cases where the model returns a low probability score, this input may be sent to a controller (may be a human moderator) which verifies and, as necessary, corrects the result. The human moderator may be used only in exceptional cases. The feedback loop feeds labeled data, auto-labeled or controller-verified, back to the model dynamically and is used as training data so that the system can improve its predictions in real-time and dynamically.

According to an embodiment of the system, the machine learning model is configured to learn using labeled data using a supervised learning, wherein the supervised learning comprises a logic using at least one of a decision tree, a logistic regression, a k-nearest neighbors, a Naïve Bayes, a random forest, a linear regression, a polynomial regression, a gradient boosting algorithm, and a support vector machine for regression. According to an embodiment of the system, the machine learning model is configured to learn from real-time data using an unsupervised learning, wherein the unsupervised learning comprises a logic using at least one of a k-means clustering, a hierarchical clustering, a hidden Markov model, and an apriori algorithm.

According to an embodiment of the system, the machine learning model has a feedback loop, wherein an output from a previous step is fed back to the machine learning model in real-time to improve performance and accuracy of an output of a next step. According to an embodiment of the system, the machine learning model comprises a recurrent neural network model.

FIG. 13B shows a structure of the neural network/machine learning model with reinforcement learning. The network receives feedback from authorized networked environments. Though the system is similar to supervised learning, the feedback obtained in this case is evaluative not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. The model may be fed with new data for testing, hence by feeding the model with data it has already predicted, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time. According to an embodiment of the system, the machine learning model has a feedback loop, wherein the learning is further reinforced with a reward for each true positive of an output of the system.

A deeper understanding of aroma benefits both industry and customers. An invention for aroma evaluation based on a gas sensor array is presented here to offer a new option other than complicated laboratory methods. The invention includes a package of hardware to collect data for odors, transfer and store data, and software to perform analysis over the collected signals using pattern recognition as well as AI methods. A case of differentiating coffee is given in this description and the method can also be applied to other odors.

According to an embodiment of the system, the microcontroller is operable to perform the aroma analysis comprising a machine learning model aided by an artificial intelligence algorithm powered by a neural network running on the cloud platform, wherein a new test case is operable to be compared to samples in a database upon request of the user. According to an embodiment of the system, the microcontroller is operable to perform the aroma analysis wherein pattern recognition results generated locally can form an ensemble with the result generated on the cloud to increase an accuracy of prediction.

According to an embodiment of the method, the microcontroller is operable to perform the aroma analysis wherein pattern recognition results generated locally form an ensemble with a result generated on the cloud platform to increase an accuracy of prediction.

Case Study 2: Detection and quantitative prediction of blood glucose level with an Electronic Nose System: Blood glucose level is an important health indicator. Non-invasive, easy-to-use glucose detection and monitoring methods and tools are desperately needed, especially for patients with diabetes. In this work, we developed a new method to quantitatively identify and analyze the blood glucose level by measuring the biomarkers in breath with an electronic nose (E-Nose) system based on a metal oxide (MOX) gas sensor array. Advanced machine-learning models have been studied and developed to precisely predict the blood glucose level based on the measurement of 41 participants for 10 days. The testing result shows that the E-Nose system, and proposed analysis models, identify blood glucose levels at an accuracy of 90.4% and a small average error of 0.69 mmol/L in blood glucose concentration. This study indicates that the E-Nose system enabled with machine learning is an efficient and precise method to achieve low-cost and non-invasive disease diagnosis. Diabetes is one of the most common diseases and there is still no cure for any type of diabetes. It is reported that 4.2 million Americans have diabetes, and 88 million people have prediabetes, accounting for 10.5% and 34.5% of US population, respectively real-time, close monitoring and treatment are necessary to manage the symptoms to a certain extent and alleviate the pain for patients. Although blood glucose measurement and monitoring are critical to diabetes patients, the regular blood sampling procedure is burdensome and painful to patients.

Many efforts have been investigated on the development of non-invasive glucose measurement methods, including radio frequency-based methods and optical methods such as Photoplethysmography (PPG). However, accurate non-invasive detection and prediction technology has not yet been developed for monitoring glucose level. In general, a gas sensor is designed as an electrical transducer to convert chemical signals into electrical signals. The sensor response, either conductance or capacitance signal, is proportional to the concentration of target gas molecules. An E-Nose consisting of gas sensors of different mechanisms and/or materials can be used to differentiate complicated mixtures of volatile organic compounds (VOCs).

In addition, much effort has been consistently focused on developing E-Nose systems for breath analysis to diagnose diseases. Different from precise gas analysis methods such as gas chromatography-mass spectrometry (GC/MS), E-Nose generates unique patterns (breath prints) while measuring the VOCs profile of human breath. The patterns, which can indicate the biomarkers of specific diseases, can be connected to the results from conventional measurement methods (e.g., blood tests) via data analysis and machine learning. E-Nose has been researched for diagnosing various respiratory diseases, such as lung cancer, asthma, and chronic obstructive pulmonary dis-ease (COPD). Some other studies revealed promising performance when using E-Nose for liver disease and kidney disease diagnosis. Moreover, a few studies performed breath analysis with E-Nose for identifying gastrointestinal disease such as colorectal cancer.

The conventional glucose measurement for diabetes diagnosis requires blood test and usually analysis under lab environment, which is invasive, time-consuming, and expensive. However, E-Nose can perform fast, simple, and non-invasive diabetes diagnosis through breath analysis, very attractive for self-monitoring blood glucose measurement. Acetone in exhaled breath, a key biomarker for diabetes, is generated when the fat is consumed for energy instead of glucose, resulting in an increase of glucose level in blood. Thus, acetone concentration in breath is reported higher for diabetes patients in comparison with healthy people. An array of gas sensors was believed to generate discriminative patterns in response to breath samples from different participants with different glucose levels. This can be a promising way for non-invasive diabetes diagnosis and glucose monitoring. A few attempts to perform diabetes diagnosis with gas sensors were made based on a limited number of medical samples. Some works used synthetic samples instead of direct data collection from patients. In addition, some previous works focused on identifying diabetes patients from healthy people with limited or no quantitative prediction of glucose levels. In comparison with qualitative analysis, quantitative glucose prediction is of greater and broader interest to health monitoring even among healthy people.

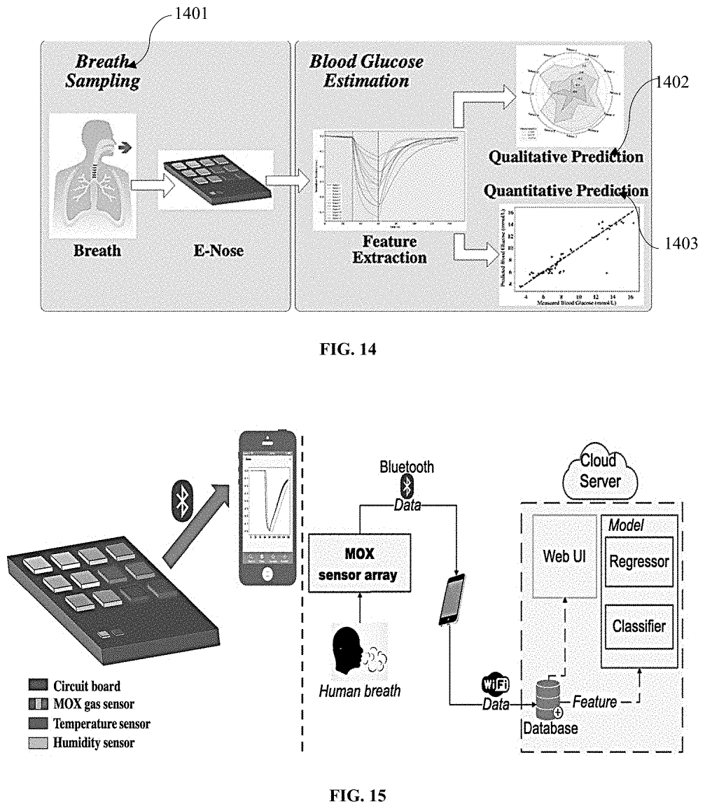

FIG. 14 shows the system for qualitative and quantitative blood glucose prediction, investigated and developed by using an E-Nose system to measure the exhaled breath. In an embodiment, methods for qualitative and quantitative blood glucose prediction are investigated and developed by using an E-Nose system to measure the exhaled breath. Firstly, a dataset is collected with the E-Nose in response to the exhaled breath of 41 participants. The data indicates strong sensor responses to breath samples from participants with high glucose level as shown at 1401. Secondly, multiple qualitative discrimination models are studied and compared on their performance of glucose classification as shown at 1402, among which gradient boosting tree (GBT) achieves the highest accuracy (90.4%). Thirdly, after a study and testing of several regression methods as shown at 1403, a hybrid quantitative glucose regressor is developed for precise prediction of blood glucose concentration with a very small average error (0.69 mmol/L).

Experimental Setup and Data Processing

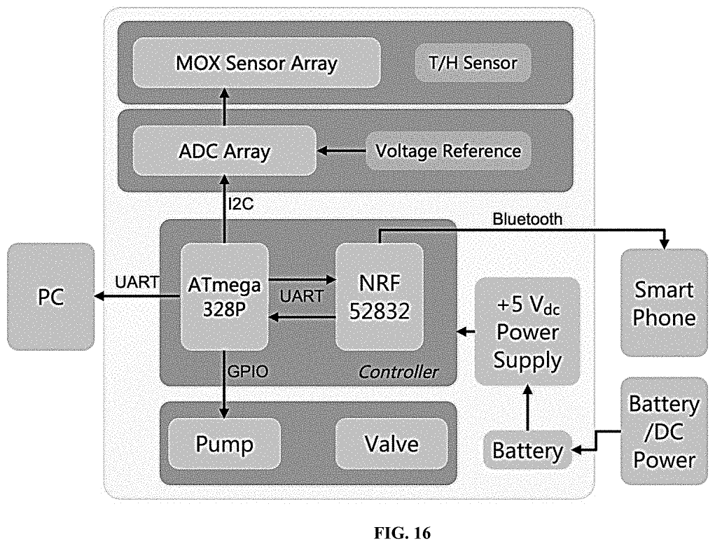

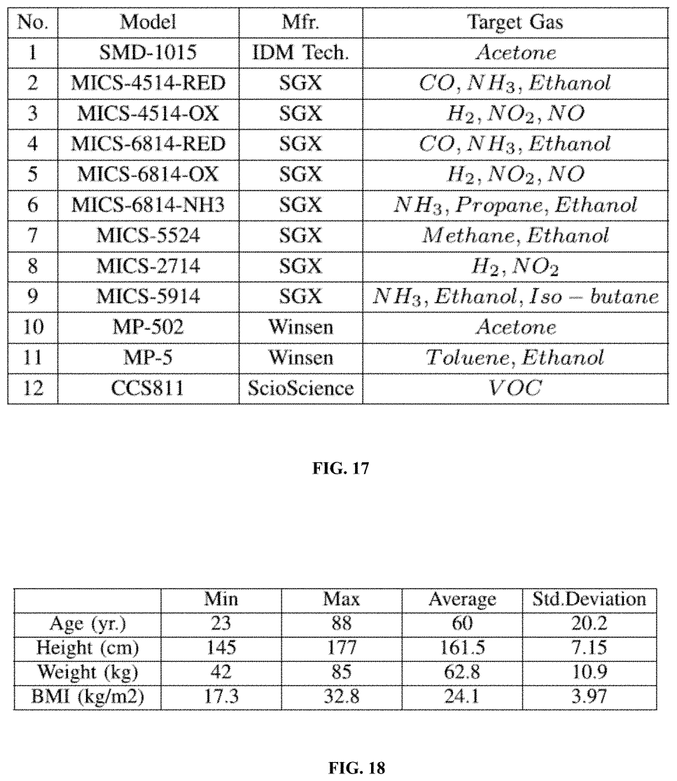

Experiment System: FIG. 15 shows the MOX gas sensor array (left); and design diagram of the system used in the experiment according to an embodiment. FIG. 16 shows the system diagram of the E-Nose device according to an embodiment. The experiment setup and system design are shown in FIG. 15 and FIG. 16. According to an embodiment, the exhaled breath was collected in a Tedlar sample bag of 1 L. During the measurement, the sample bag was connected to the gas chamber with an array of 12 commercial MOX gas sensors. FIG. 17 shows the MOX gas sensors used in the e-nose system according to an embodiment. According to an embodiment, temperature and humidity sensors were integrated in the chamber to monitor and stabilize the measurement conditions because the temperature and moisture disturb the conductance of MOX gas sensors. The chamber temperature was modulated by the heating voltage supplied to the heaters of sensors, and the heating voltage was controlled by a PID controller taking chamber temperature as the feedback. A humidity controller module was deployed to stabilize the chamber humidity to adjust the humidity of intake air. The intake air humidity was modulated by a second PID controller based on the moisture measured at the chamber. Our customized signal conditioning circuit captured the resulting sensor response signals and sent them to smartphones over Bluetooth®. Once the measurement was finished, data would be saved and transmitted to a cloud server and stored in a database. A web user interface is developed on the server for visualization of rough data and prediction results. Machine learning classification and regression models were implemented and trained for blood glucose level prediction using the data in the server. The parameters of machine learning models are obtained from training and saved on the server for future predictions. During testing, unknown breath data samples will be transmitted to the server and prediction will be achieved based on the parameters.

The E-Nose system used in our experiment has two microprocessors, among which ATmega328P is used for general Input/Output (I/O) controlling and NRF52832 dedicated to Bluetooth® communication. In each measurement cycle, ATmega328P controls the behavior of the pump and valve and interfaces with gas sensors through an array of analog-to-digital converter (ADC) attached to its I2C bus. ATmega328P transmits data to NRF52832 through software universal asynchronous receiver-transmitter (UART) while optionally communicating with a desktop PC through hardware UART. The entire system can be powered by an external power supply or a battery of 7.4V.

Breath Sample Dataset Preparation: The breath samples were collected and measured by the Department of Respiratory Diseases at the First Affiliated Hospital of Hainan Medical University, Haikou, China. The dataset comprises 210 measurements obtained from 41 participants (21 females and 20 males). The participants were continuously diagnosed and measured for at least five days, and each participant was sampled once each day. The breath samples were collected from the participants at 7:30 am following a regular fasting procedure. Participants were instructed to inhale to total lung capacity and then fully exhale into the Tedlar sample bag through a breathing mouthpiece attached to a tube. A carbon filter was connected between the mouthpiece and the sample bag to remove the particles from the ambient environment. FIG. 18 shows the physical information of recruited participants with evenly distributed age and body mass index (BMI) according to an embodiment. The blood glucose level was measured from a blood test as venous plasma. The glucose levels from all measurements ranged from 3.3 mmol/L to 17.4 mmol/L according to our dataset. In addition, to minimize the temperature variation, the breath sample bags were kept for 10 minutes to reach room temperature before being tested by the E-Nose system. The E-Nose system was pre-heated for 20 minutes before measurements so that the chamber temperature was stabilized at 60° C. according to the onboard temperature sensors. In addition, the flow rate of the mechanic pump in E-Nose was calibrated to 150 sccm.

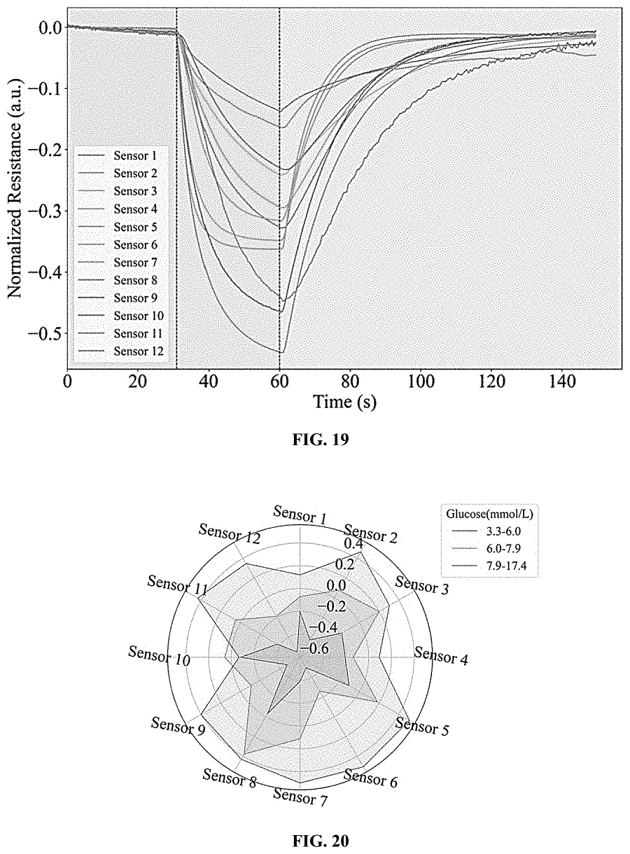

FIG. 19 shows the electrical signals of all 12 sensors were measured simultaneously in response to a breath sample and recorded by the E-Nose according to an embodiment. Each measurement cycle, lasting 150 s, consisted of three phases: (1) baseline phase, (2) response phase, and (3) recovery phase. During the baseline phase, air was inhaled into the gas chamber for 30 s to clean the chamber and generate stable sensor baselines. From 30 s to 60 s, the breath samples were inhaled into the chamber and sensors responses were measured and recorded. After that, air was injected into the chamber to exhaust the breath from 60 s to 150 s. To ensure the consistent sensor baseline values for all measurements, an interval of 3 minutes between two consecutive measurements was applied, during which the pump was set to full speed at 250 sccm to purge the breath in chamber and pipelines.

Data Pre-Processing: Data pre-processing steps were performed to transform sensor signals into feature vectors for each sample. The measured voltage drop across sensors were converted into resistance values accordingly. In addition, discrete wavelet transformation (DWT) was used to remove the noise in the resistance signals. Baseline normalization was used to minimize the resistance drifting for the gas sensors. As shown in equation (1), the normalized resistance is a ratio between resistance change and the baseline resistance, where Rₐᵣₒₘₐ is the measured sensor resistance and R_{baseline} is the baseline sensor resistance.

$${R}_{i} = \frac{{R}_{aroma} - {R}_{b aseline}}{{R}_{b aseline}} - \qquad Equation ( 1 )$$

As the sensor array in the E-Nose system had 12 different MOX gas sensors, the feature combinations might be quite complex, causing high feature dimensions and overfitting. Therefore, we used only the maximum responses as features. Moreover, normalization was performed across all data samples to ensure every sensor has the same weight in contribution to the prediction.

Machine Learning Models for Blood Glucose Prediction

Blood Glucose Range Classification: For the purpose of calibration and parameter training, the glucose levels of all participants were measured daily via blood test before the collection of breath samples. The measurement results were labeled into three classes: below 6.0 mmol/L, 6.0 mmol/L-7.9 mmol/L, and above 7.9 mmol/L. The ranges were selected to have a balance of numbers among the three classes. FIG. 20 shows the median values of normalized maximum sensor responses versus blood glucose ranges for all sensor features according to an embodiment. As the blood glucose level increased, the values of sensor response increased, indicating the acetone concentration in the breath is proportional with the glucose level in the blood.

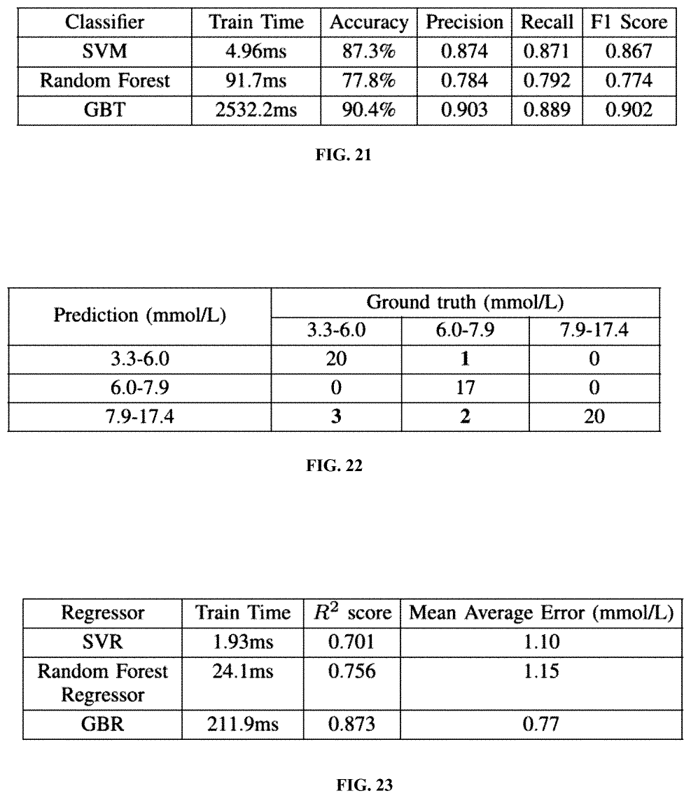

To avoid possible overfitting on the sensor data, three most frequently used classifiers, support vector machine (SVM), random forest and gradient boosting tree (GBT), have been applied to study the analysis and prediction of blood glucose level in this work. SVM was reported to have good performance on small datasets with high dimensions. The ensemble learning methods, such as Random Forest and GBT, can effectively reduce the risk of overfitting. In this study, each of the three classifiers was trained and cross-validated with 147 training samples and tested on the remaining 63 samples. FIG. 21 shows the testing set that was selected by randomly choosing 30% of the samples on each day according to an embodiment. The predicted results were also summarized in FIG. 21. Accuracy standards for the percentage of correctly classified samples; precision is the ratio between the number of true positive samples and samples classified as positive; recall represents the ratio between the number of true positive samples and all positive samples; and F1 score is the harmonic mean of precision and recall.

The results showed that the prediction of blood glucose level using feature extraction of E-Nose signals was quite effective and precise. The prediction accuracy was 87.3%, 77.8% and 90.4% for the three classifiers, respectively. SVM was the most efficient model with the fastest training and good classification accuracy. GBT achieved the best classification accuracy at a sacrifice of the training time. FIG. 22 listed the detailed prediction results of GBT according to an embodiment. It is indicating that samples between 7.9 mmol/L and 17.4 mmol/L were easily distinguished from the other two groups while a few samples of low glucose were misclassified as high glucose. The satisfactory results in classification from the three models paved the road for quantitative glucose prediction with high precision.

Regression Models for Prediction of Blood Glucose Concentration: Quantitative analysis on blood glucose will be of great interest for diabetes diagnosis and monitoring. In this work, three different frequently used regressors, SVM regressor (SVR), Random Forester regressor and gradient boosting regressor (GBR), were used and trained with the same training/testing split as the above-discussed classification according to an embodiment. FIG. 23 shows the regression performance according to an embodiment. It indicates that prediction precision increases at the cost of training time.

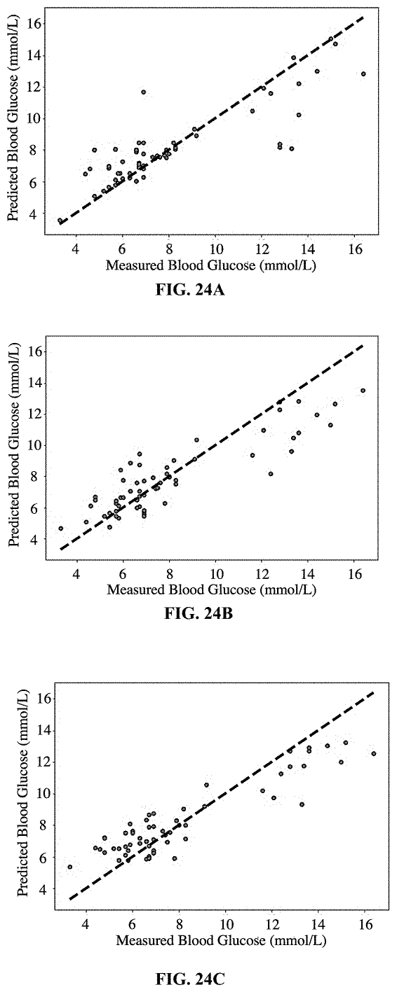

FIG. 24A shows the glucose level predicted using exhaled breath versus ground truth obtained by blood tests for SVR regressor. FIG. 24B shows the glucose level predicted using exhaled breath versus ground truth obtained by blood tests for Random Forest regressor. FIG. 24C shows the glucose level predicted using exhaled breath versus ground truth obtained by blood tests for GBR regressor. The predictions on the test set generated by the three regressors are plotted in FIGS. 24A, 24B, and 24C. Overall, GBR uses the longest training time (about 0.2 s) but is able to achieve the best prediction performance, 0.77 mmol/L mean average error (MAE) in comparison with the other two regressors. To further improve the prediction performance, a hybrid regressor combining both regression and classification will be developed.

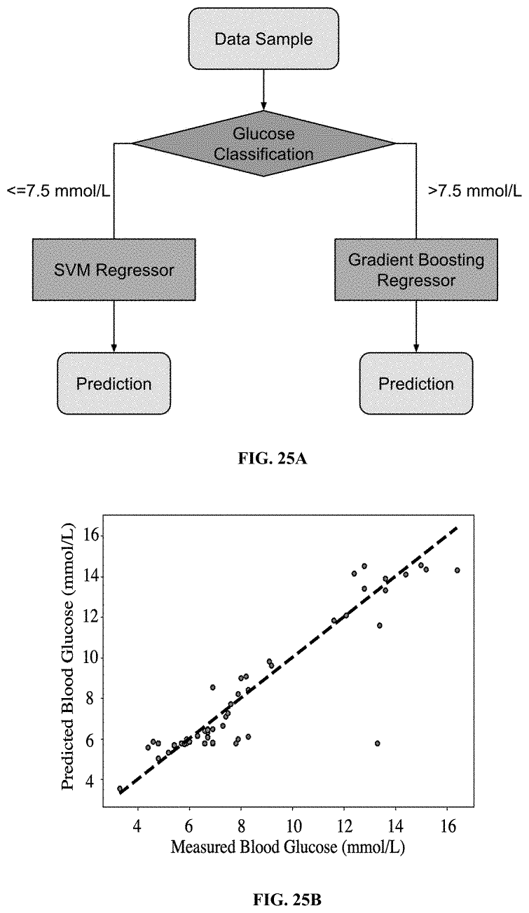

FIG. 25A shows a hybrid regressor decision making diagram where the GBT classifier was trained to determine the blood glucose range of data samples; both SVR and GBR were trained to provide refined prediction on different ranges according to an embodiment. As shown in FIG. 25A a hybrid regressor was developed by performing SVM regression or GBR immediately following GBT classification. The GBT classifier was to determine whether the blood glucose level of the incoming data sample was above or below 7.5 mmol/L. Depending on the classifying result, two regressors were trained on different glucose level ranges (above or below 7.5 mmol/L) to refine the prediction. FIG. 25B shows prediction of the hybrid regressor versus ground truth according to an embodiment. As shown in FIG. 25B the hybrid regressor model exhibited a remarkable improvement in glucose prediction (0.845 R2 score and very small MAE of 0.69 mmol/L) compared to the three single regressors. The prediction is evenly and nearly distributed around the ground truth obtained in blood tests.

The performance improvement is attributed to the fact that the hybrid regression algorithm perfectly solved the issue of imbalanced distribution of blood glucose values in the dataset. For instance, in this study, larger amounts of data samples lie in the range of 6.0 mmol/L-8.0 mmol/L than in other ranges. In this case, the three single regressor models over-weighted the samples of lower glucose values while under-weighting samples of higher glucose values, causing lower prediction precision in some ranges of values. This work reported a study of E-Nose system and advanced machine learning models to precisely identify and quantitatively predict the blood glucose levels based on exhaled breath. The array of gas sensors exhibited increasing response magnitude to the breath samples of participants with higher blood glucose levels. Three frequently used classifiers have been studied and compared, showing effective identification on the unknown samples with satisfactory accuracy. Three regression models were evaluated on the performance of quantitative blood glucose level prediction based on exhaled breath. GBR achieved the best performance with a small MAE of 0.77 mmol/L. We developed a hybrid regressor model to further refine the predictions in different blood glucose ranges. The hybrid regressor achieved the remarkable prediction performance with a very small MAE of 0.69 mmol/L. The results indicated that precise and effective blood glucose prediction could be achieved by an E-Nose with an array of low-cost MOX gas sensors and machine learning models designed to address imbalanced dataset. This study showed an attractive, low-cost solution for non-invasive diabetes diagnosis and health monitoring.

According to an embodiment, the system can be used for coffee quality examination. The system can predict coffee samples' bitterness and sourness based on their odors.

According to an embodiment, the system can be used for differentiating original and fake fragrance. The system can determine whether a given fragrance sample is fake, given an original sample as the reference.

### INCORPORATION BY REFERENCE

All references, including granted patents and patent application publications, referred herein are incorporated herein by reference in their entirety.
U.S. Patent Application Publication US20160161459A1, titled “Apparatus for Detection and Delivery of Volatilized Compounds and Related Methods”
U.S. Patent Application Publication US20180120278A1, titled “Apparatus for Volatile Organic Compound (VOC) Detection”
U.S. Patent Publication U.S. Ser. No. 10/330,624B2, titled “Metal Oxide Gas Sensor Array Devices, Systems, and Associated Methods”
U.S. Patent Application Publication US20200088705A1, titled “Method of Operating Gas Sensors and Corresponding Device, Sensor and Program Product”
U.S. Patent Application Publication US20190317079A1, titled “System and Method for Volatile Organic Compound Detection”
U.S. Patent Application Publication US20200271605A1, titled “Gas Sensing Device and Method for Operating a Gas Sensing Device”

1-75. (canceled)

76. A system, comprising:
a specialized device comprising:
a flow sub-system configured for sampling a gas sample;
a gas chamber having a gas sensor array comprising a configurable sensor interface;
wherein the specialized device is operable to collect an aroma signal from the gas sample;
a microcontroller comprising a processor and a memory, operable to:
digitalize the aroma signal to obtain aroma data;
store and transfer the aroma data;
perform an aroma analysis on the aroma data; and
provide a feedback to a user;
wherein the system is an aroma evaluation system operable to detect a target aroma in real-time; and
wherein the system is operable to interface to at least one of a cloud platform and a smartphone.

77. The system of claim 76, wherein the specialized device is capable of sniffing aroma through at least a first inlet and a second inlet wherein the first inlet is for intake of the target aroma and the second inlet is for intake of air to exhaust the target aroma in the specialized device, wherein the first inlet and the second inlet are controlled by a solenoid valve wherein the solenoid valve is a 3-way solenoid valve.

78. The system of claim 76, wherein the flow sub-system comprises a flow controller, a pump, and a gas flow sensor, wherein the flow sub-system comprises a configurable air flow scheme through the flow controller for the sampling of the gas sample, wherein the configurable air flow scheme comprises a micro air flow sensor operable to monitor a flow rate of the gas sample through the system.

79. The system of claim 76, wherein the configurable air flow scheme is operable to multiple patterns of sampling air which creates distinct features for the target aroma, and wherein the flow controller is a software reading feedback from the gas flow sensor and calculating a corresponding control signal for the pump.

80. The system of claim 76, wherein the flow rate is controlled to generate multiple patterns including sinusoid, pulses, and even simulated human breath.

81. The system of claim 76, wherein the specialized device comprises a heat modulated sampling.

82. The system of claim 76, wherein the aroma signal is sampled and captured by the microcontroller on the specialized device wherein the aroma signal is further conditioned, wherein signal conditioning comprises denoising with discrete wavelet transform, normalizing based on a sensor baseline, extracting features based on signal's magnitude, gradient during sensor response and recovery, and curve fitting parameters.

83. The system of claim 76, wherein the microcontroller is operable to store the aroma data locally.

84. The system of claim 76, wherein the microcontroller is operable to perform the aroma analysis, and a result of identification of the aroma and to return the result to the user in real-time, wherein the aroma analysis comprises detecting information of a breed and the target aroma, and wherein the breed comprises one of a different types of the target aroma and concentration of certain chemicals in the target aroma.

85. The system of claim 76, wherein the microcontroller is operable to perform the aroma analysis comprising a pattern recognition algorithm for a target discrimination which is deployed on a standalone device.

86. The system of claim 76, wherein the configurable sensor interface is designed to accommodate different interfaces, mechanisms, and sizes of the gas sensor array.

87. The system of claim 76, wherein the configurable sensor interface is a three-level interface comprising a control board, a sensor board, and an interface board, wherein the interface board comprises configurable blocks to create a customizable layout to accommodate various sensor combinations and placement schemes.

88. The system of claim 76, wherein the configurable sensor interface further comprises at least one of a humidity sensor, a temperature sensor and an onboard power light-emitting diode (LED) to indicate working status.

89. The system of claim 76, wherein the feedback to the user comprises information of a breed and the target aroma, wherein the breed comprises one of a different types of the target aroma and concentration of certain chemicals in the target aroma, wherein the feedback to the user is through an interactive display.

90. A method, comprising:
obtaining an aroma signal from a gas sensor, comprising:
starting a pump of a flow sub-system to let air flow into a gas chamber of a specialized device comprising a gas sensor array until reaching a steady state;
switching a solenoid valve to intake a target aroma which is a gas sample for a certain amount of time;
stopping the pump;
shutting down the gas chamber with at least two solenoid valves around it, such that molecules in the target aroma fully interact with the gas sensor array;
switching the solenoid valve to bring in air by the pump to exhaust the target aroma remaining;
reading the aroma signal from the gas sensor;
digitalizing the aroma signal to aroma data;
storing and transferring the aroma data;
performing an aroma analysis on the aroma data; and
providing feedback to a user;
wherein the method is an aroma evaluation method operable to detect the target aroma in real-time; and
wherein the method is executable on a device that is operable to interface to at least one of a cloud platform and a smartphone.

91. The method of claim 90, wherein a configurable air flow scheme is operable to multiple patterns which create distinct features for the target aroma.

92. The method of claim 90, wherein the aroma signal is operable to be analyzed using pattern recognition and artificial intelligence algorithms.

93. The method of claim 90, wherein the aroma analysis comprises information of a breed and the target aroma, wherein the breed comprises one of a different type of the target aroma and concentration of certain chemicals in the target aroma.

94. The method of claim 90, wherein the method is operable to perform the aroma analysis comprising an artificial intelligence algorithm powered by a neural network running on the cloud platform, wherein a new test case is operable to be compared to samples in a database upon request of the user.

95. A non-transitory computer-readable medium having stored thereon instructions executable by a computer system to perform operations comprising:
obtaining an aroma signal from a gas sensor, comprising:
starting a pump of a flow sub-system to let air flow into a gas chamber of a specialized device comprising a gas sensor array until reaching a steady state;
switching a solenoid valve to intake a target aroma which is a gas sample for a certain amount of time;
stopping the pump;
shutting down the gas chamber with at least two solenoid valves around it, such that molecules in the target aroma fully interact with the gas sensor array;
switching the solenoid valve to bring in air by the pump to exhaust the target aroma remaining;
reading the aroma signal from the gas sensor;
digitalizing the aroma signal to aroma data;
storing and transferring the aroma data;
performing an aroma analysis on the aroma data; and
providing feedback to a user;
wherein the instructions executable by the computer system are configured for an aroma evaluation operable to detect the target aroma in real-time; and
wherein the instructions are executable on a device that is operable to interface to at least one of a cloud platform and a smartphone.
